(19) United States
(12) Patent Application Publication
(10) Pub. No.: US 2021/0000675 A1
(43) Pub. Date: Jan. 7, 2021

(54) Traction and Decompression Therapy for the Cervical Spine

(71) Applicants: Scot Leslie Johnson, Tampa, FL (US); Saleem Musallam, Tampa, FL (US)

(72) Inventors: Scot Leslie Johnson, Tampa, FL (US); Saleem Musallam, Tampa, FL (US)

(21) Appl. No.: 16/922,718

(22) Filed: Jul. 7, 2020

Related U.S. Application Data
(60) Provisional application No. 62/871,182, filed on Jul. 7, 2019.

(51) Int. Cl. A61H 1/02

(52) U.S. Cl. CPC A61H 1/0222; A61H 1/0296

(57) ABSTRACT

A traction and decompression device for use in spinal therapy. The device includes a contoured headrest having a contour shaped and sized to fit a patient's head. The device also includes an adjustment mechanism having a base plate securable to an external structure. The adjustment mechanism also includes a headrest plate secured to the contoured headrest. The device may be incorporated into a spinal therapy bed or other apparatus. In certain embodiments, the device may be attached to a motorized actuator.

### BACKGROUND OF THE PRESENT DISCLOSURE

The human spine consists of four distinct regions of vertebra which are (generally) connected by intervertebral discs. These consist of the Cervical, Thoracic, Lumbar, and Sacral regions. The Cervical region consists of locations labeled “C0”, “C1”, “C2”, “C3”, “C4”, C5”, “C6”, and “C7”. The Thoracic region consists of locations labeled “T1”, “T2”, “T3”, “T4”, “T5”, “T6”, “T7”, “T8”, “T9”, “T10”, “T11”, and “T12”. The Lumbar region consists of locations labeled “L1”, “L2”, “L3”, “L4”, and “L5”. For the sake of traction and decompression therapy of the human spine, the only relevant location of the Sacral region is labeled “S1”.

The vertebra are bone structures which vary according to the segment and region of the backbone. Most vertebra are connected by intervertebral discs which allow slight movement of the vertebra and act as “shock absorbers” for the spine. The structure of the intervertebral discs is complex, however their function is dependent primarily of the nucleus pulposus (the central portion of the intervertebral disc) which performs the function of load distribution within the spine. Intervertebral discs are labeled as, for example, the intervertebral disc located between C4 and C5 is labeled “C4-5”.

There is a normal curvature of the spine, the so called ‘S-Curve’. There is a normal ‘lordosis’ (forward curves) of the cervical and lumbar spinal regions. There is a normal ‘kyphosis’ (backward curves) of the thoracic and sacral spine.

C0 is also known as the occiput or occipital bone, the flat bone that forms the back of the head/skull. C1 is also termed the “Atlas”, and C2 the “Axis”. There are no intervertebral discs between C0, C1, and C2. C1 and C2 form a unique set of articulations that provide a great degree of mobility for the skull. C1 serves as a ring or washer that the skull rests upon and articulates in a pivot joint with the dens or odontoid process of C2. Approximately 50% of flexion extension of the neck happens between the occiput and C1; 50% of the rotation of the neck happens between C1 and C2.

### BRIEF DESCRIPTION OF THE DRAWINGS

The foregoing disclosure will be best understood and advantages thereof made most clearly apparent when consideration is given to the following detailed description in combination with the drawing figures presented. The detailed description makes reference to the following drawings:

FIG. 1 shows an illustration of the human spine;

FIG. 2 shows an illustration of the skull and cervical spine;

FIG. 3 shows a human patient placed supine on a treatment bed;

FIG. 4 shows manual manipulation of the cervical spine;

FIG. 5 shows a traction device;

FIG. 6 shows a mechanical apparatus with occipital posts retracted;

FIG. 8 shows a mechanical apparatus with occipital posts extended;

FIG. 8 shows a mechanical apparatus placed on the upper mattress of a treatment bed;

FIG. 9 shows the mechanical apparatus connected to slides;

FIG. 10 shows an electro-mechanical automated mechanism placed outside of the upper mattress;

FIG. 11 shows a rotational motor connected to the mechanical apparatus;

FIG. 12 shows a contoured headrest from a first viewpoint;

FIG. 13 shows a contoured headrest from a second viewpoint;

FIG. 14 shows a patient on a therapy bed, with the patient's skull resting in the contoured headrest;

FIG. 15 illustrates a close-up of the skull rotated backwards within the contoured headrest and held in place by a forehead strap;

FIG. 16 shows a mechanism for adjusting the position of the contoured headrest;

FIG. 17 illustrates the assembly of FIG. 16 in differences suitable to true decompression;

FIG. 18 illustrates a version of FIG. 17, with the headrest plate resting, and with the contoured headrest attached to it;

FIG. 19 shows the system of FIG. 18 with the headrest plate and contoured headrest extended towards the proximal end of the system;

FIG. 20 illustrates the patient's skull positioned appropriately for true decompression within the contoured headrest;

FIG. 21 illustrates the system of FIG. 20, with some differences;

FIG. 22 illustrates the rotational motor, at a fixed distance from the distal end of the upper mattress, though raised in elevation relative to the surface of the upper mattress;

FIG. 23 demonstrates a close-up view of FIG. 22 centered on observance of the alignment of C2 through C5;

FIG. 24 illustrates the rotational motor, raised as in FIGS. 22 and 23, the handle tab attached similarly to the rotational motor creating an angle between the hinge plate and base plate;

FIG. 25 demonstrates a close-up view of FIG. 24;

FIG. 26 demonstrates the current state of technology for solid state laser modules;

FIG. 27 illustrates both the focused single point laser emission as well as the left and right sides of the cone of laser light produced by slightly defocusing the laser emission;

FIG. 28 demonstrates a laser manifold designed to house the laser modules and permit their optical emission to pass through to the cervical spine;

FIG. 29 illustrates an array of eight laser modules positioned below the laser manifold;

FIG. 30 demonstrates an assembly which is one embodiment of the present invention;

FIG. 31 illustrates an electronic means for controlling the laser modules in one embodiment of the present invention;

FIG. 32 describes the assembly of the system of FIG. 30, the laser control PCB, and an optional battery pack;

FIG. 33 describes the system of FIG. 32, with eight focused laser beams emitting from the eight laser modules installed;

FIG. 34 describes the system of FIG. 32, with eight defocused laser beams emitting from the eight laser modules installed;

FIG. 35 describes components which can be utilized to form a portable housing for the laser system of FIG. 32;

FIG. 36 illustrates exploded views of the laser system of FIG. 32 being secured into the shell formed of the manifold cover and base platform;

FIG. 37 shows the system of FIG. 36 fully assembled;

FIG. 38 demonstrates the portable laser device placed appropriately on a simple mechanical apparatus;

FIG. 39 shows the portable laser device of FIG. 37, with a modification of a port or connection by which a signal may be delivered from outside of the portable laser device;

FIG. 40 demonstrates the profile of the forces applied during both traction and decompression therapy;

FIG. 41 shows the contoured headrest of FIG. 12 with certain modifications;

FIG. 42 shows an exploded view of the method of securing the laser system of FIG. 32 onto the headrest plate; and

FIG. 43 shows the system of FIG. 42 with the addition of the modified contoured headrest of FIG. 41.

### DETAILED DESCRIPTION OF THE DISCLOSURE

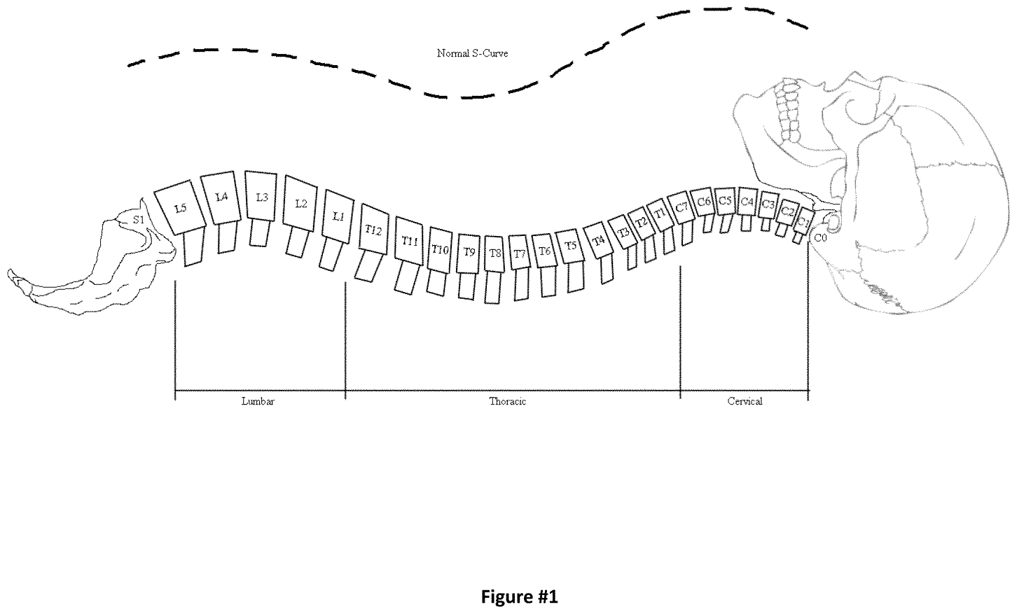

Refer to FIG. 1 for an illustration of the Skull, Cervical, Thoracic, Lumbar and Sacral spine. The normal S-curve is indicated, as are the regions of the spine. Each vertebral bone relevant to traction and decompression therapy is labeled.

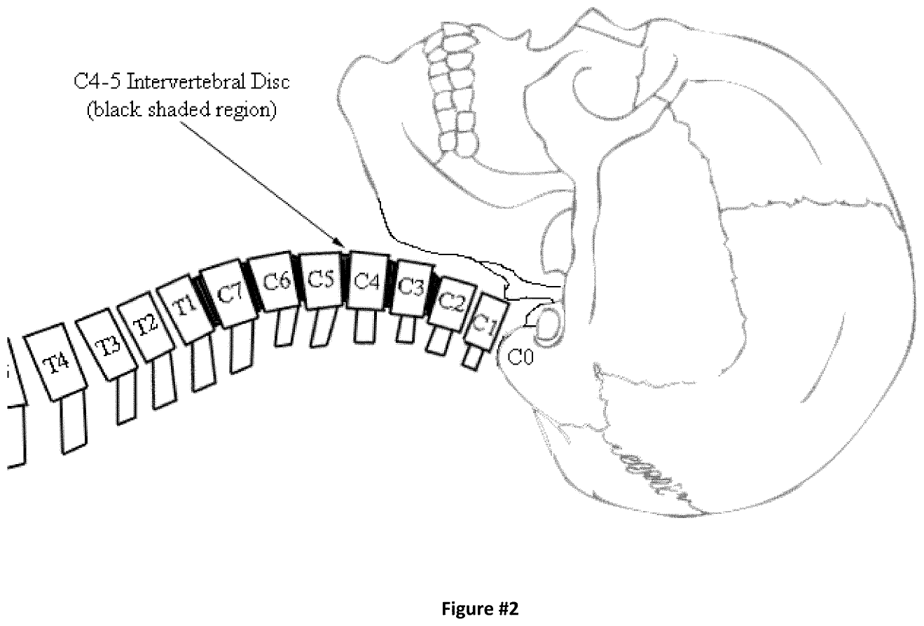

Refer to FIG. 2 for an examination of the Skull and Cervical spine, with adjoining intervertebral cervical discs. The intervertebral discs are indicated by shaded-BLACK structures (an example of which is pointed out, the intervertebral disc connecting C4 and C5).

Various non-optimal states of the spine may be treated with either traction or decompression. Regarding compressive forces normally experienced by the spine, the intervertebral discs rely on the annulus fibrosus (an outer ring of the intervertebral disc) and the nucleus pulposus which distributes compressive forces in all directions to minimize the compression. The annulus fibrosus consists of fibrocartilage surrounding the nucleus pulposus which distributes compressive forces in normal day-to-day use. The annulus fibrosus in and of itself functions to withstand some portion of compressive forces in normal day-to-day use. There are several conditions which can reduce the (anti-compressive) function of the annulus fibrosus and nucleus pulposus.

‘Herniation’ is the term which describes deformation of the annulus fibrosus which allows the gel-like nucleus pulposus to protrude, distorting muscular function and/or putting pressure on nearby nerves. Commonly referred to as ‘slipped disc’, the disc is not physically slipped. It may bulge, usually in one direction. By unloading the intervertebral disc, the bulge may be pulled back into position and the intervertebral disc can heal and function normally.

‘Degeneration’ is a condition in which the intervertebral discs, either through repeated stress injuries, aging, or genetic predisposition, reduced function of the intervertebral disc can lead to back pain.

While there are a variety of conditions which lead to back pain, including herniation and degeneration, restoration of the function of the intervertebral disc through physical therapy, traction and decompression can and often does lead to healing of the spine and normal spinal function with regards to day-to-day function and quality of life.

In the case of traction, a continuous distractive force is applied to the spine, and in the case of the present invention, the cervical spine, to reduce intervertebral disc compressive force and allow for period the intervertebral discs time to heal. This technique has certain drawbacks. The paraspinal muscles, those muscles that connect the spinal vertebra, tend to contract over time during the therapy which lessens and may even exacerbate the condition of the spine. Great care must be taken by either the patient (as this form of therapy is readily available to patients for self-treatment) or the healthcare provider for patients seeking treatment.

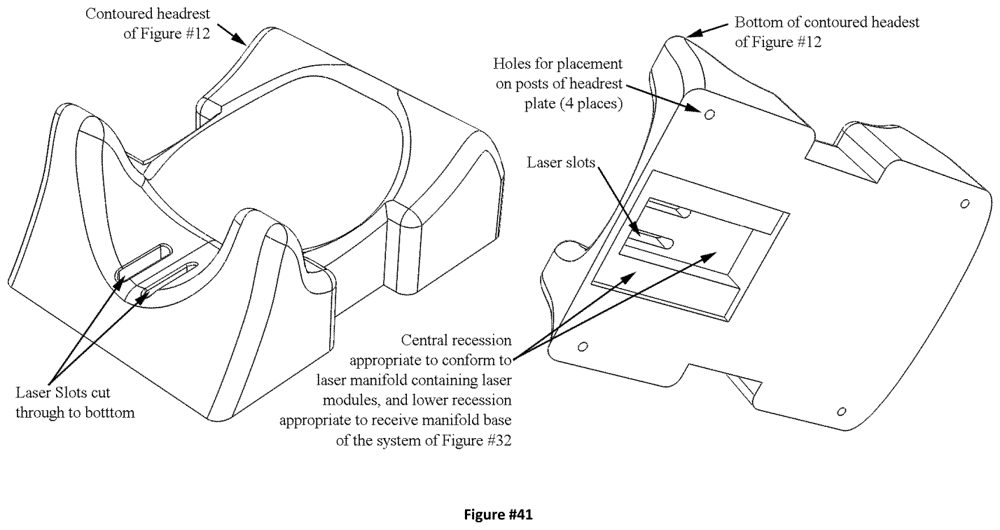

In the case of spinal decompression, there are two philosophies that exist. The first philosophy, which for the purpose of this provisional patent application will be referred to as ‘basic decompression’, relies on the cyclical application of distraction forces applied to the spine. In this respect, the distractive forces applied to the cervical spine are cycled between distraction and zero applied force. Refer to FIG. 40, described later in this provisional patent application. Rather than applying continuous distractive force, the distractive force is applied for a period of time, then relaxed, then reapplied, the cycle being repeated during the treatment. The advantage of basic decompression is the distractive force applied is temporary, then relaxed, thereby reducing the likelihood that the paraspinal muscles will contract and limit or prevent unloading of the intervertebral disc.

The second philosophy that is applied to decompression therapy involves both cyclical distractive force and specific alignment of the cervical intervertebral discs/vertebra such that distractive forces applied pull ‘through’ and to the specific disc or discs that are to be treated. For the purpose of this provisional patent application, this form of decompression will be referred to as ‘true decompression’. True decompression therapy represents the most advanced non-surgical form of unloading of compressive forces and healing of the intervertebral disc(s).

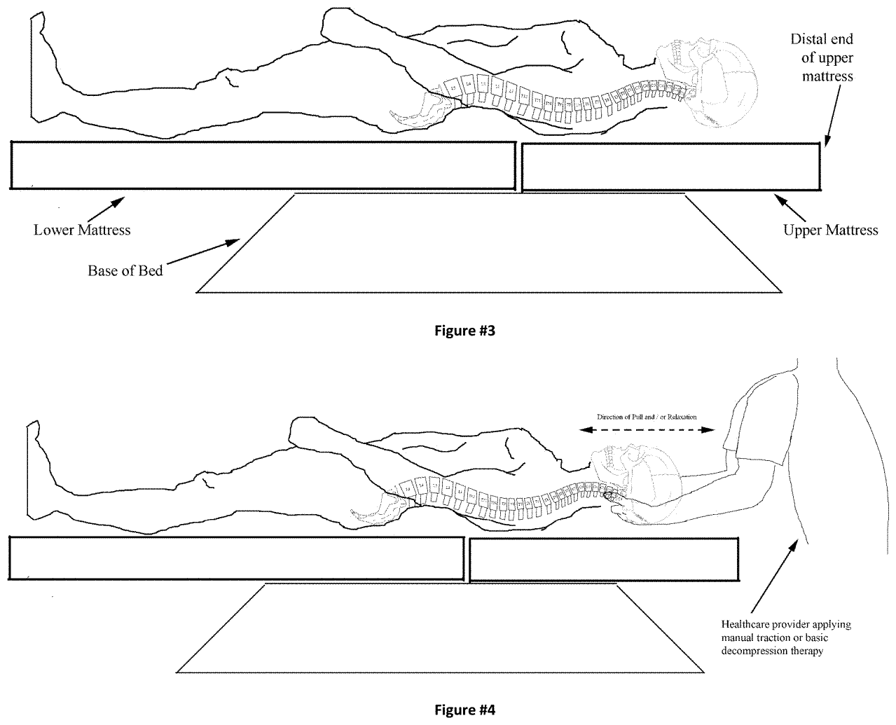

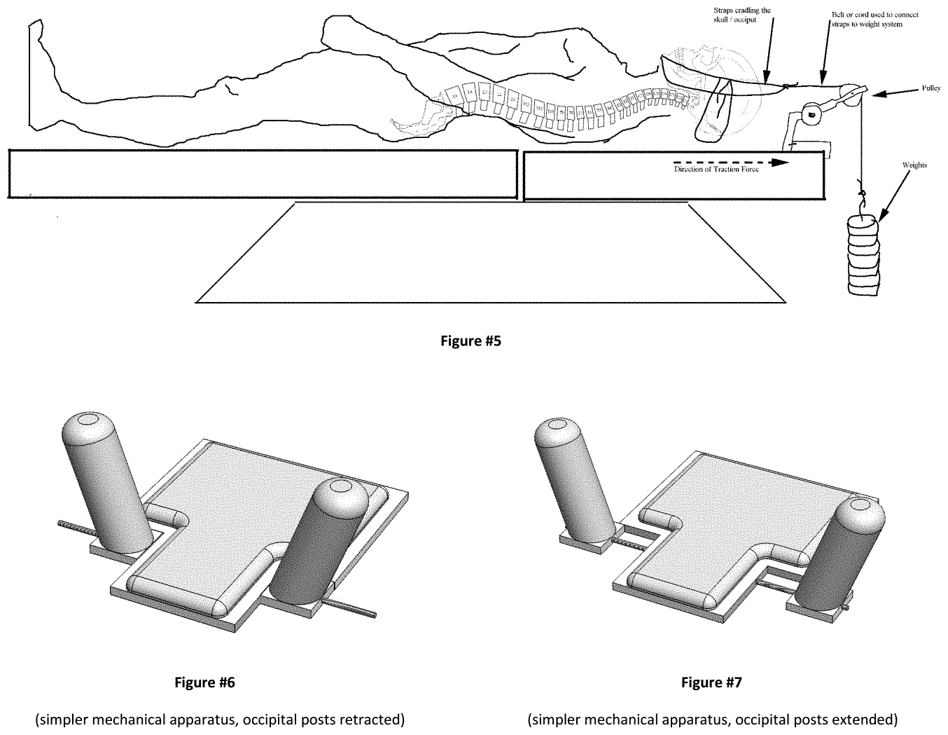

Typically, the human patient is placed supine on a treatment bed (refer to FIG. 3). The most basic form of cervical traction or decompression therapy relies on “manual” manipulation of the cervical spine (refer to FIG. 4). The healthcare provider cups the base of the spine, referred to as the “occiput” or “C0”. The healthcare provider then pulls the skull in an attempt to provide traction, or cycles the pulling force in an attempt to provide basic decompressive therapy. As will be described further in this application, true decompression therapy almost always involves a medical apparatus to maintain a certain posture of the skull and C0, C1 and C2.

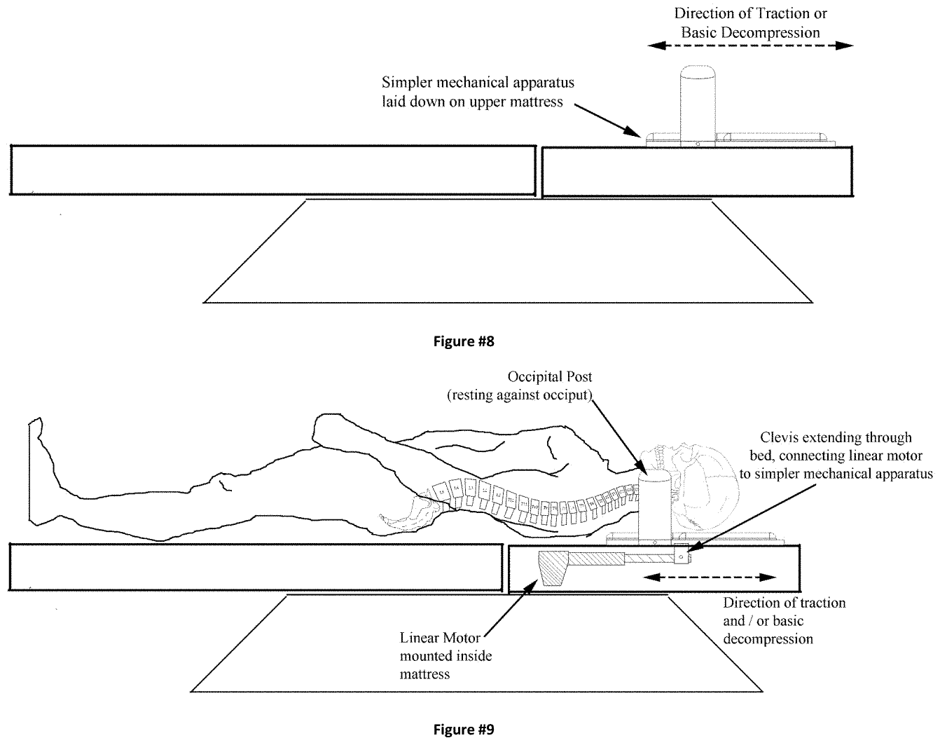

Various mechanical apparatus have been developed to aid in either traction or decompression therapy. The very most basic relies on straps which cup the bottom of the chin and typically either or both the forehead and occiput. These straps connect to a pulley typically via a belt or cord, which may be placed over a chair (if the patient is seated), or the end of a bed (if the patient is lying on a bed), and beyond the pulley the belt or cord is connected to one or more weights. These simplest devices typically provide pure traction. Refer to FIG. 5.

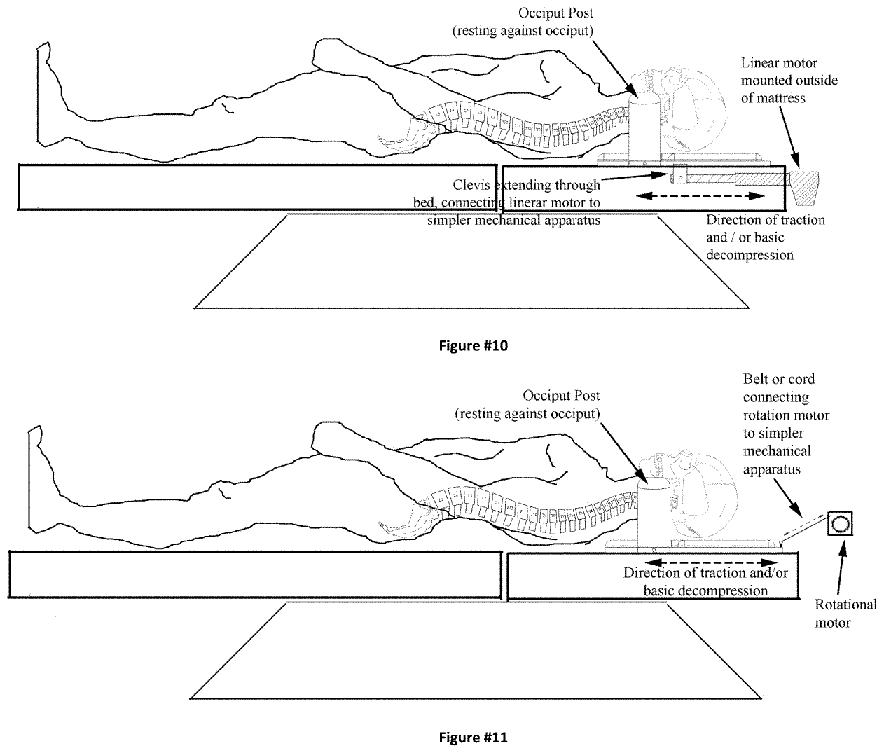

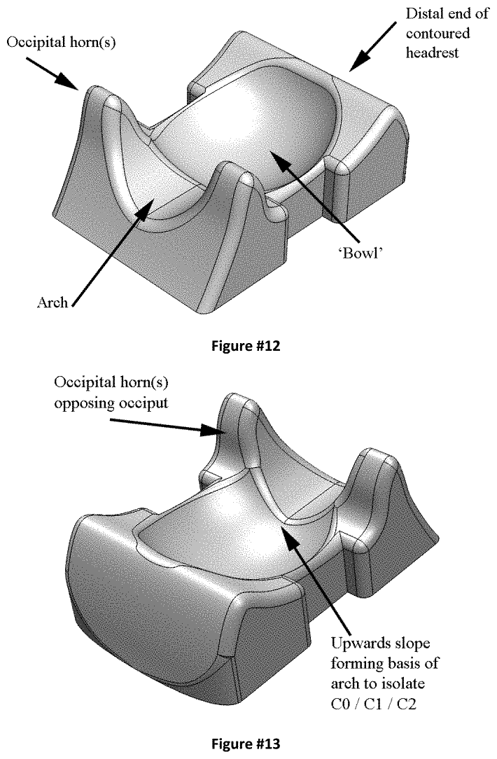

One other of the simpler mechanical apparatus involves a flat, often cushioned surface upon which the back of the patient skull is laid. Often, two cushioned posts are included which can be manipulated inwards or outwards to conform to the diameter of the patient neck and provide a back-stop for the occiput on either side of the neck. Refer to FIGS. 6 and 7. As described, this simpler form of apparatus is typically either laid down on the upper end of a bed mattress (as a means to manipulate the cervical spine manually), or is integrated into the upper end of a mattress. In the integrated version, this form of apparatus is typically mounted on one or more slides underneath the mattress, integrated via a channel cut through the upper end of the mattress, such that this form of apparatus may slide back and forth along the length of the mattress enabling either traction or basic decompression (refer to FIG. 8).

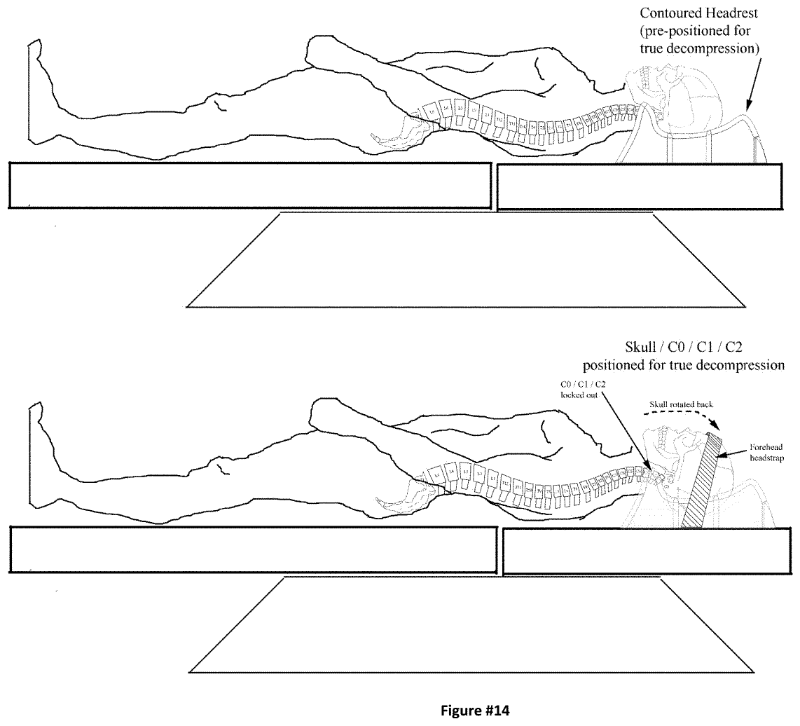

In the case of the simpler mechanical apparatus integrated into an upper end of a mattress and connected to slides or other device which allows lengthwise movement of the apparatus, an electro-mechanical automated means (for example a linear motor) may also be integrated such that the headrest can be extended towards the distal end of the upper mattress and retracted, providing traction (by pushing) and/or basic decompression (by pushing and pulling) (refer to FIG. 9). Further other embodiments may utilize an electro-mechanical automated means (for example a linear motor) be placed outside of the upper mattress, again providing traction (by pulling) and/or basic decompression (by pulling and pushing) (refer to FIG. 10).

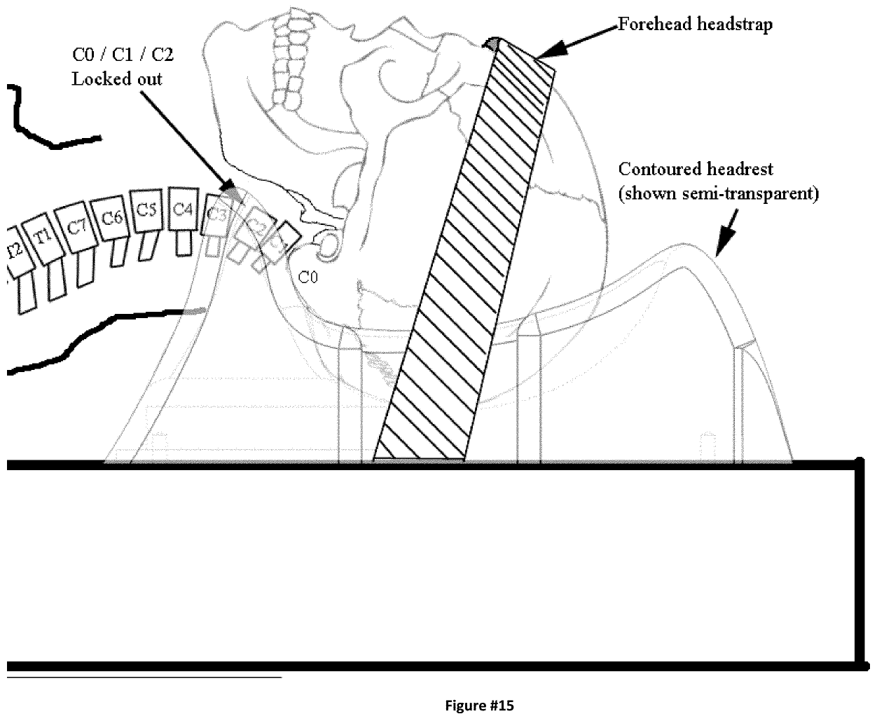

One additional means of utilizing the simpler mechanical apparatus to perform traction or basic decompression therapy is to connect to the apparatus a belt or cord which is then attached to a rotational motor mounted behind the distal end of the upper mattress, the rotation motor retracting and releasing the belt or cord to provide traction or decompression therapy (refer to FIG. 11).

As previously described, ‘true decompression’ therapy involves both cyclical distractive force and specific alignment of the cervical intervertebral discs/vertebra such that distractive forces applied pull ‘through’ and to the specific disc or discs that are to be treated. Alignment of the cervical vertebra, and thus the cervical intervertebral discs, requires specific alignment of most importantly C0, C1 and C2. As described previously, there are no intervertebral discs between C0, C1, and C2. C1 and C2 form a unique set of articulations that provide a great degree of mobility for the skull. C1 serves as a ring or washer that the skull rests upon and articulates in a pivot joint with the dens or odontoid process of C2. Approximately 50% of flexion extension of the neck happens between the occiput and C1; 50% of the rotation of the neck happens between C1 and C2. Before alignment of the vertebra C3, C4, C5, C6, C7 (and potentially T1) can occur, and therefore intervertebral discs C2-3, C3-4, C4-5, C5-6, C6-7, and C7-T1, the extreme range of the C0/C1/C2 vertebra must be accounted for and reduced and/or eliminated.

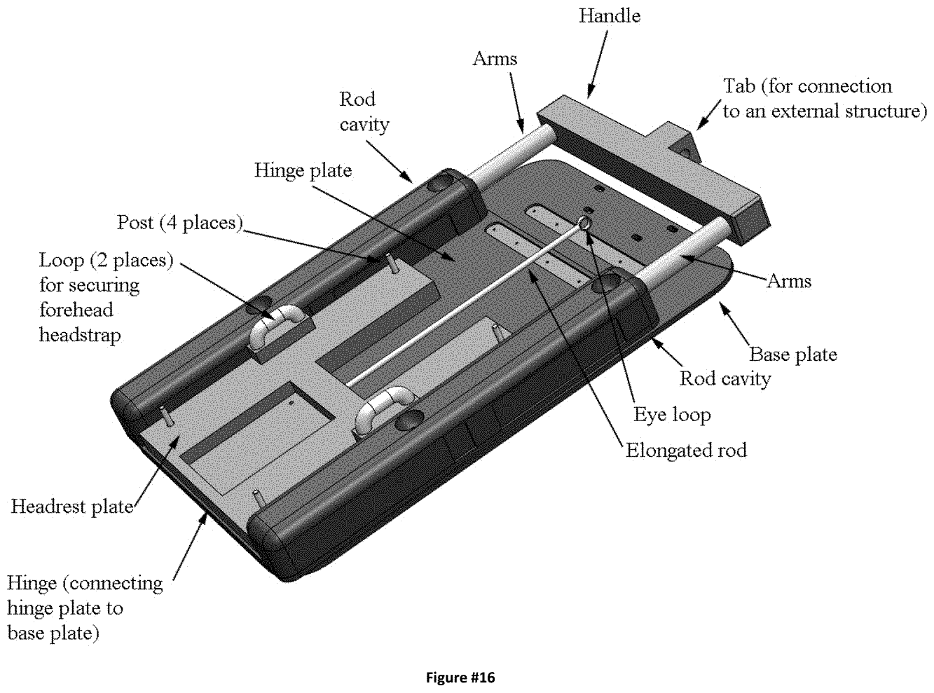

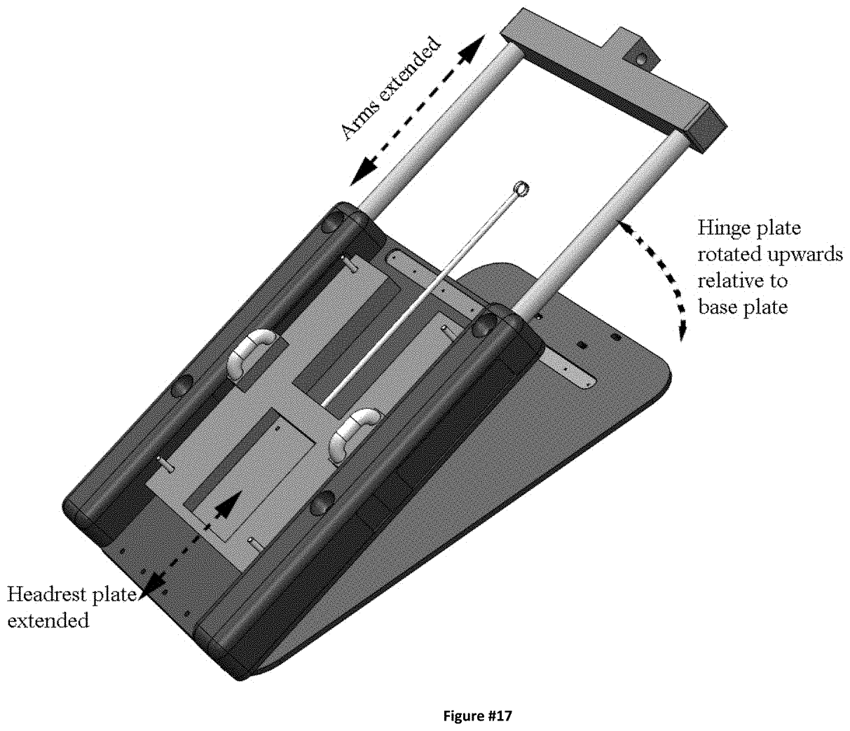

As part of one embodiment of the present invention, a unique “contoured headrest” is presented which can allow for comfortable positioning and restraint of the skull, C0, C1 and C2 such that targeted, true decompression can proceed. Refer to FIGS. 12 and 13.

The “contoured headrest” should be made of a material that provides comfort and some give, such as an open or closed cell foam, for example a ‘memory foam’. Silicone rubbers, of a softer hardness, durometer or ‘shore’ rating, as well as materials such as foam, may be injection-molded reliably and repeatedly. Additionally, these materials may be over-molded onto a base, such as a metal plate, which may be used to secure the finished contoured headrest to an apparatus which allows alignment and connection to an automated electro-mechanical motor to facilitate traction or decompression forces. The contoured headrest may be made in non-permanent physical layers, to allow for variations in patient morphology and patient tolerance to traction or distractive forces (higher and lower durometer or ‘shore’ ratings).

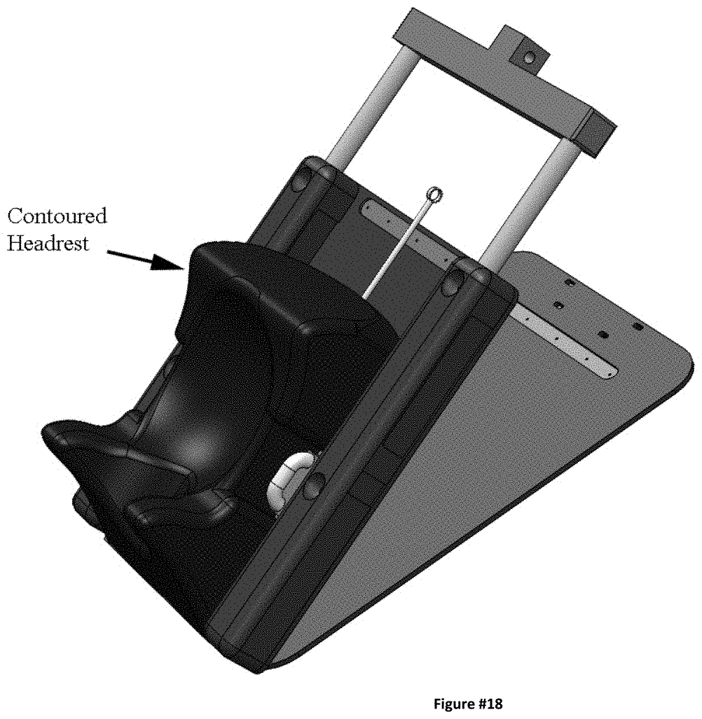

FIGS. 12 and 13 illustrate one such embodiment of the present invention. The portion of the contoured headrest which would face the distal end of the upper mattress contains a ‘bowl’ which conforms to the back of the skull. Two ‘occipital horns’ are positioned such that when the patient skull is resting in the bowl the occiput is positioned against the occipital horns. An arch between the bowl containing the skull and the proximal end of the headrest which falls beyond the occipital horns is designed to isolate, when properly applied, the C0/C1/C2 joint(s). The skull and thus C0, C1 and C2 must be flexed such that no other flexion/rotation is possible. This can be accomplished by manipulation of the skull within the contoured headrest and securing the skull there with a forehead strap. Refer to FIG. 14 for illustration of the patient upon first resting onto the headrest, and secondarily when the skull, C0/C1/C2 are manipulated to isolate movement in these regions and held in place by a forehead headstrap proper for true decompression.

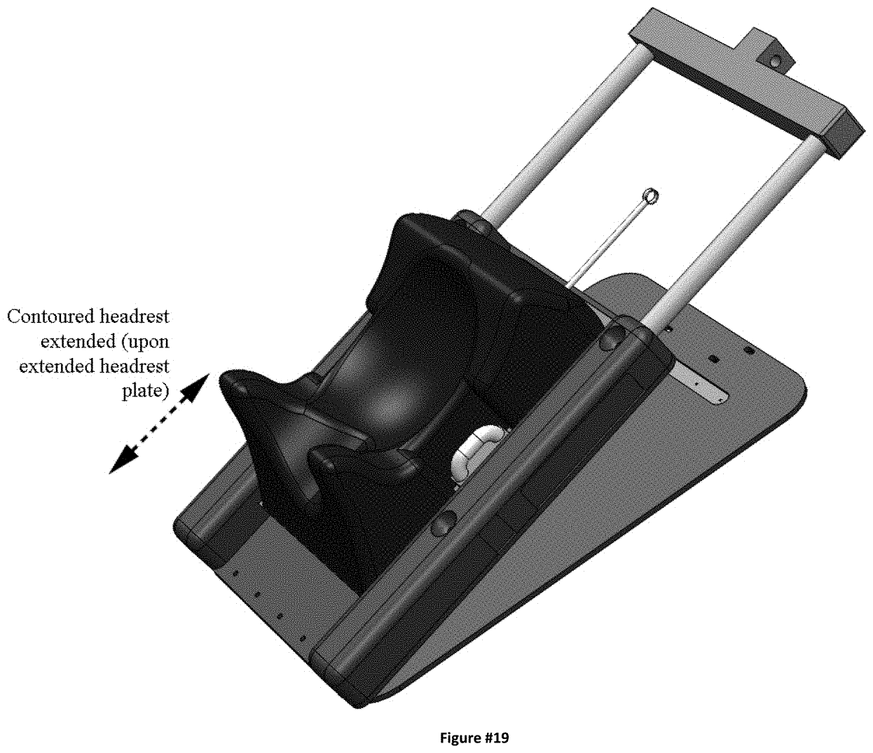

FIG. 15 illustrates a close-up of the skull rotated backwards within the ‘bowl’ of the contoured headrest and held in place by a forehead strap, and vertebra C1 and C2 locked out of any further motion, proper for true decompression.

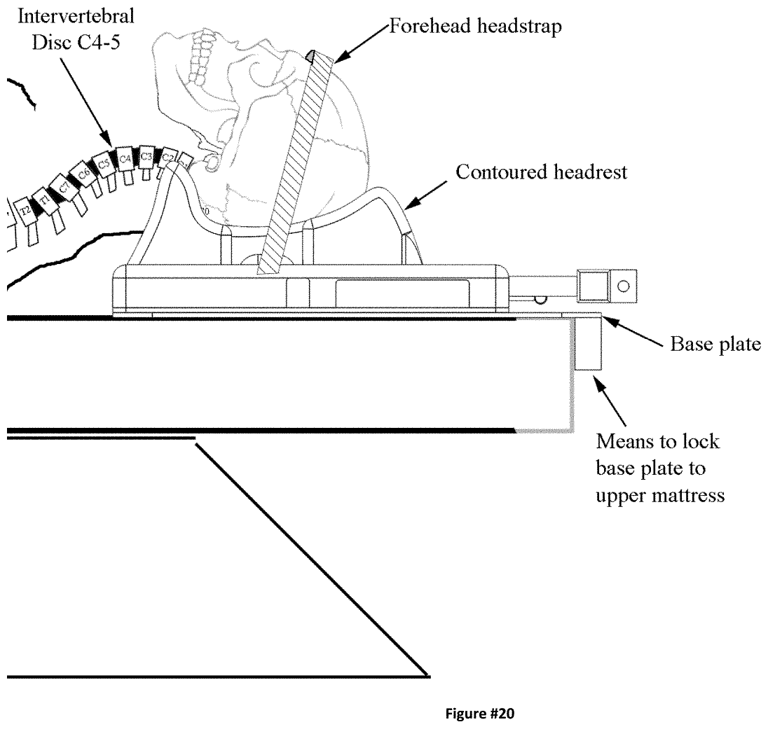

To accomplish cervical traction, basic decompression, or true decompression, a means by which the contoured headrest, one embodiment of the present invention, can be both moved towards and away from the distal end of the upper mattress must be established. Refer to FIG. 16.

A machined or injection molded plate, made of perhaps aluminum, delrin, or other appropriate plastic or material, with posts appropriate to fix the ‘contoured headrest’ to the plate, is shown in FIG. 16. This plate, and for the sake of this embodiment of the present invention will be referred to as the “headrest plate” hereon, contains loops fixed to its sides for securing a forehead headstrap. Additionally, an elongated rod, made of some sturdy material such as stainless steel, extends from the distal end of the headrest plate ending in an eye loop (for attachment to a belt or cord). The headrest plate is attached to a secondary plate by means of mechanical slides (not shown in the figure or in this provisional application), the secondary plate hereon referred to as the “hinge plate”. The slides allow the headrest plate to slide lengthwise along the hinge plate. Two elongated rounded rectangular prisms (made perhaps of machined delrin or other suitably machineable or injection-moldable lightweight material), hereon referred to as the “rod cavities”, are secured to the hinge plate on either side of the headrest plate. The rod cavities contain a circular bore extending from the distal end back to nearly the proximal end of the rod cavities. Two tubes, ideally made of stainless steel (the “arms”), are permitted to slide into and out of the circular bores and are connected by a ‘handle’ (ideally made of a machined delrin or injection molded material) which contains a tab designed for connection to an external structure. The hinge plate is mounted on yet a final plate, in this embodiment and for ease of reference referred to hereon as the “base plate”, is mounted to the proximal end of the base plate by means of a hinge, such as a piano hinge (not shown). This hinge attachment allows the hinge plate, headrest plate and anything attached to these to rotate upwards about the hinge.

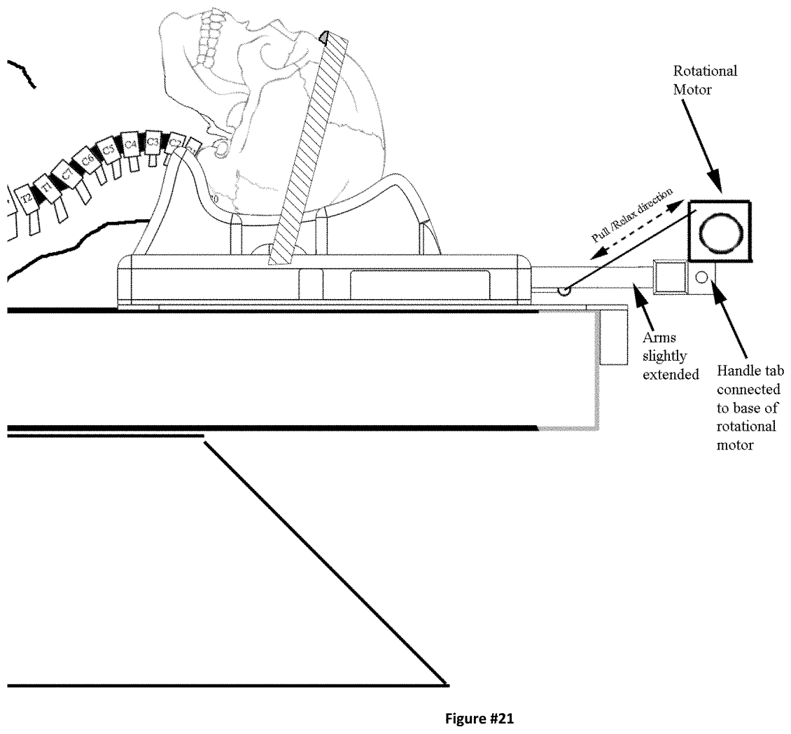

FIG. 17 illustrates the assembly of FIG. 16 in differences suitable to true decompression. Firstly, the hinge plate is rotated upwards at an angle via the hinge relative to the base plate. Secondly, the “arms” and handle are extended towards a yet undefined external structure, and thirdly the headrest plate is shown extended upon its slides relative to the resting state shown in FIG. 16.

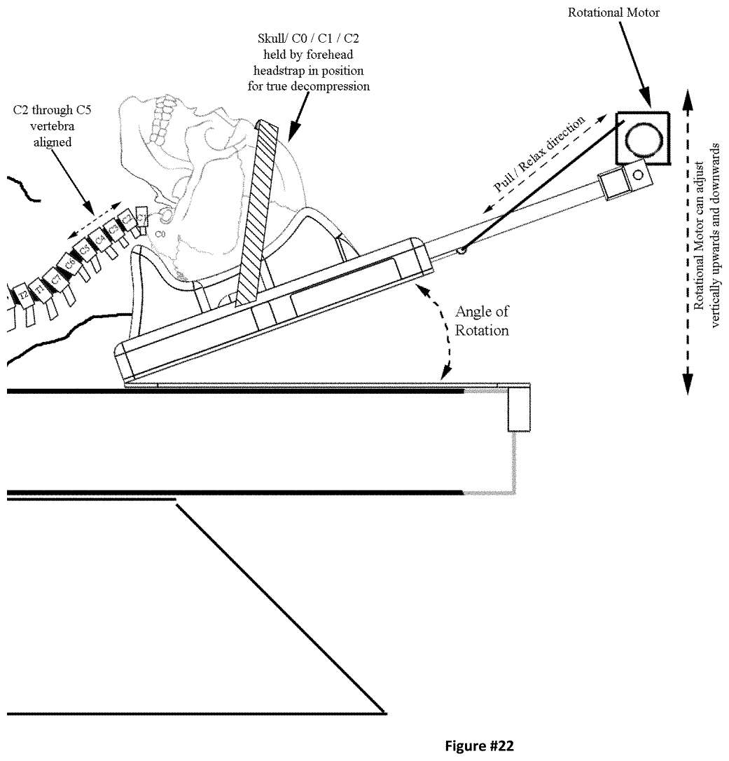

FIG. 18 illustrates a version of FIG. 17, with the headrest plate resting (not extended), and with the contoured headrest attached to it. In one embodiment of the present invention, the contoured headrest is a suitable material as previously described, overmolded onto a metal plate which contains four holes positioned such that upon placement onto the headrest plate align with the posts, securing it in place onto the headrest plate. FIG. 19 shows the system of FIG. 18, however with the headrest plate and contoured headrest extended towards the proximal end of the system.

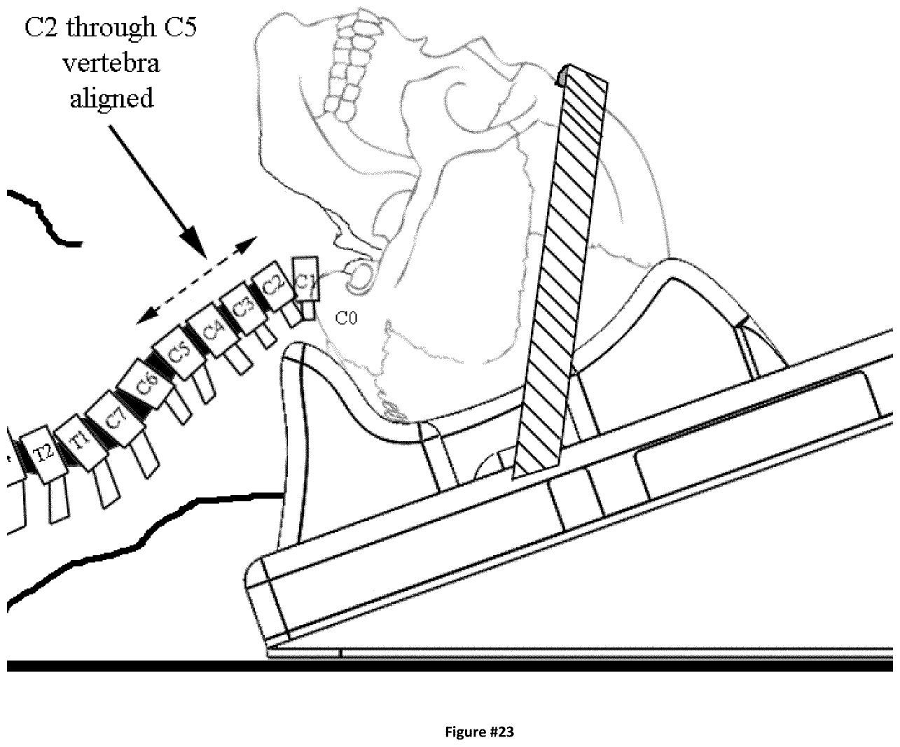

Refer to FIG. 15. The skull/C0/C1/C2 are positioned within the headrest and secured there by the forehead strap appropriate (as previously described) for true decompression. FIG. 20 illustrates the patient's skull/C0/C1/C2 positioned appropriate for true decompression within the contoured headrest. The cervical intervertebral discs are shown shaded black, between the cervical vertebra, and for the sake of illustration the intervertebral disc C4-5 is pointed out. The contoured headrest is shown secured to the system of FIGS. 16 through 19. The system of FIGS. 18 and 19 is shown laid upon the upper mattress of a bed upon which the patient lay. The base plate is flat upon the upper mattress at its distal end. A means of connection to the distal end of the upper mattress which may be accomplished by several means, for example a slotted component that slides over a post at the end of the distal mattress (as in a temporary securing) or as a more permanent means secured via hardware (screws/nuts/bolts etc.).

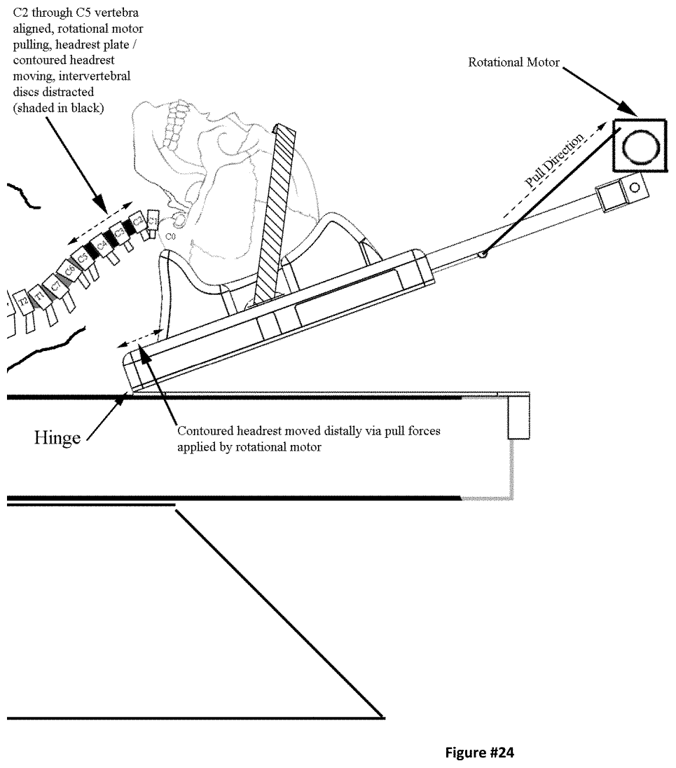

FIG. 21 illustrates the system of FIG. 20, with some differences. The arms are extended beyond the distal end of the mattress, the handle tab of the handle connecting the arms fixed via an appropriate means (not shown) to a rotational motor. A belt or cord is attached to the eye loop located at the end of the elongated rod (which is attached to the headrest plate), the other end of said belt or cord is connected to the rotational motor. The rotational motor may rotate such that the belt or cord retracts and ‘pulls’ the headrest plate (which contains the contoured headrest, and thus the patient skull, vertebra and intervertebral discs) creating a distractive, unloading force on the skull and cervical structures. The rotational motor may rotate in the opposite direction, unloading the belt or cord, and thus relaxing the skull and cervical structures (this ‘pull’ and ‘relax’ function indicated in the Figure). In this respect, the system of FIG. 21 is performing basic decompression.

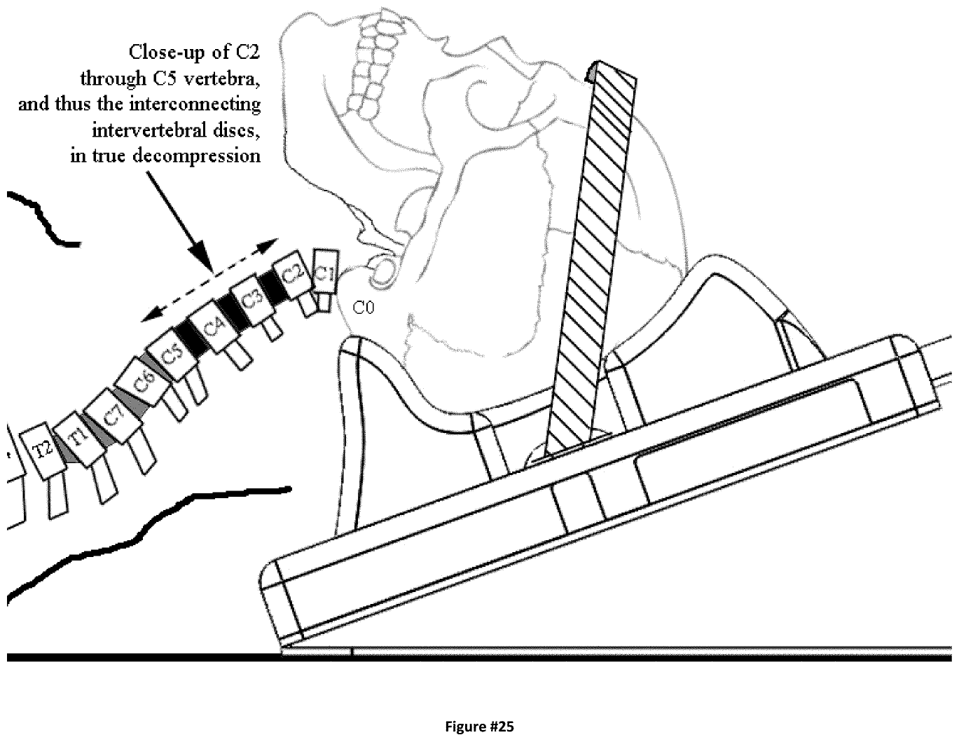

FIG. 22 illustrates the rotational motor, at a fixed distance from the distal end of the upper mattress, though raised in elevation relative to the surface of the upper mattress. In this embodiment of the present invention, the rotational motor can be manually or automatically (such as by an electro-mechanical means, for example a linear motor) raised or lowered. With the handle tab connected to the rotational motor (for example, the base of the rotational motor), the hinge plate and those parts connected to the hinge plate (including the skull and cervical components) are raised along with the rotational motor, the arms extending as necessary to maintain this function. The angle of rotation of the hinge plate relative to the fixed base plate (via the hinge) is indicated in the figure. The skull/C0/C1/C2 are retained via the forehead headstrap in this embodiment. The belt or cord connected between the eye loop and rotational motor are illustrated, as are the direction of pull or relaxation required for decompression. In this Figure, and at this angle of rotation of the hinge plate, the hinge plate and base plate are arranged such that the vertebra C2 through C5 are aligned, as is shown in the Figure.

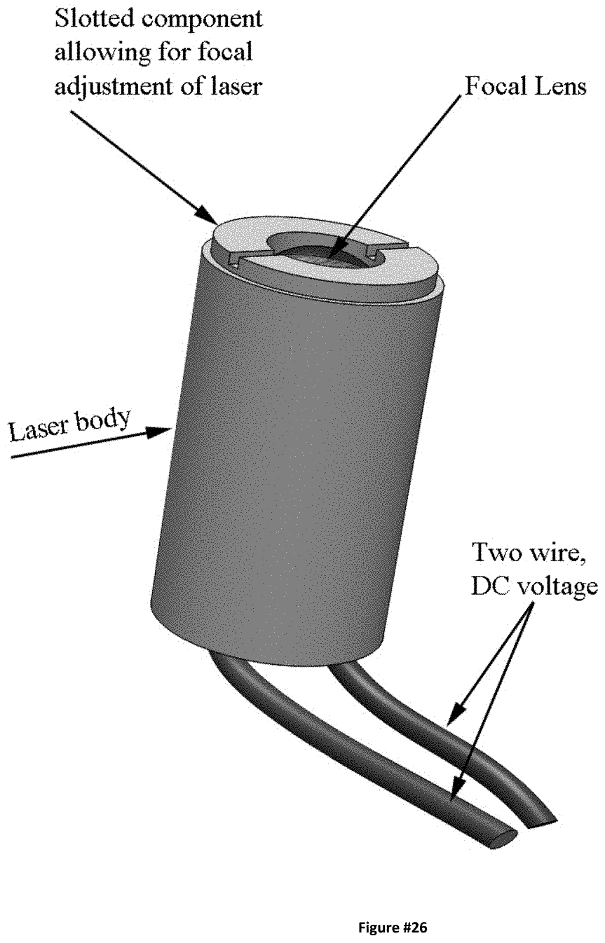

FIG. 23 demonstrates a close-up view of FIG. 22 centered on observance of the alignment of C2 through C5. The normal lordosis of the cervical spine between C2 through C5 is reduced to a straight alignment (shown in the Figure), whereas the normal lordosis between C5 through T1 is preserved at this angle of rotation. The intervertebral discs, as previously described, are shown as shaded in black, those between C2 through C5 are clearly aligned.

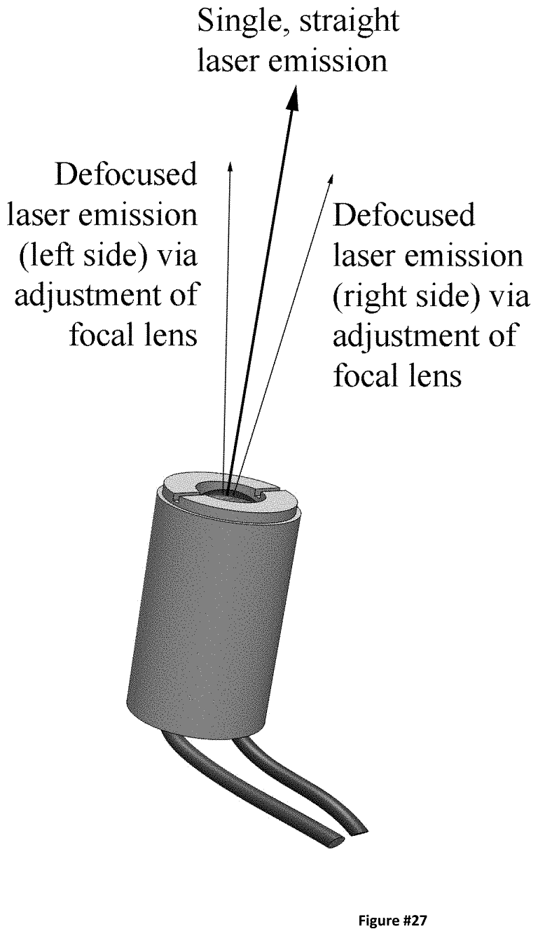

FIG. 24 illustrates the rotational motor, raised as in FIGS. 22 and 23, the handle tab attached similarly to the rotational motor creating an angle between the hinge plate and base plate. The belt or cord connected to the eye loop and rotational motor is being pulled by the rotational motor (shown in the Figure). This pulling force has moved the headrest plate (upon its slides connected to the hinge plate) forward relative to the proximal end of the hinge plate, also illustrated in the Figure. Through this unloading of the cervical spine vertebra, and thus the intervertebral discs C2-3 through C4-5, the intervertebral discs are elongated (shown elongated in the illustration, shaded in black), and those intervertebral discs beginning with C5-6 through C7-T1 are shaded in gray and are not elongated (due to the existing lordosis). This is a form of true decompression, and based on the discussion regarding decompression, the cycling of distractive forces between distraction and relaxation, will achieve maximal rehydration of the intervertebral discs with minimal paraspinal muscle contractions. Assuming the intervertebral disc to be treated is C4-5, it is necessary to pull through C2-3 through C4-5 to achieve a targeted true decompression treatment of C4-5, and to cycle the distraction (‘pulling’) force and to relax it (again, refer to FIG. 40 described further in the document).

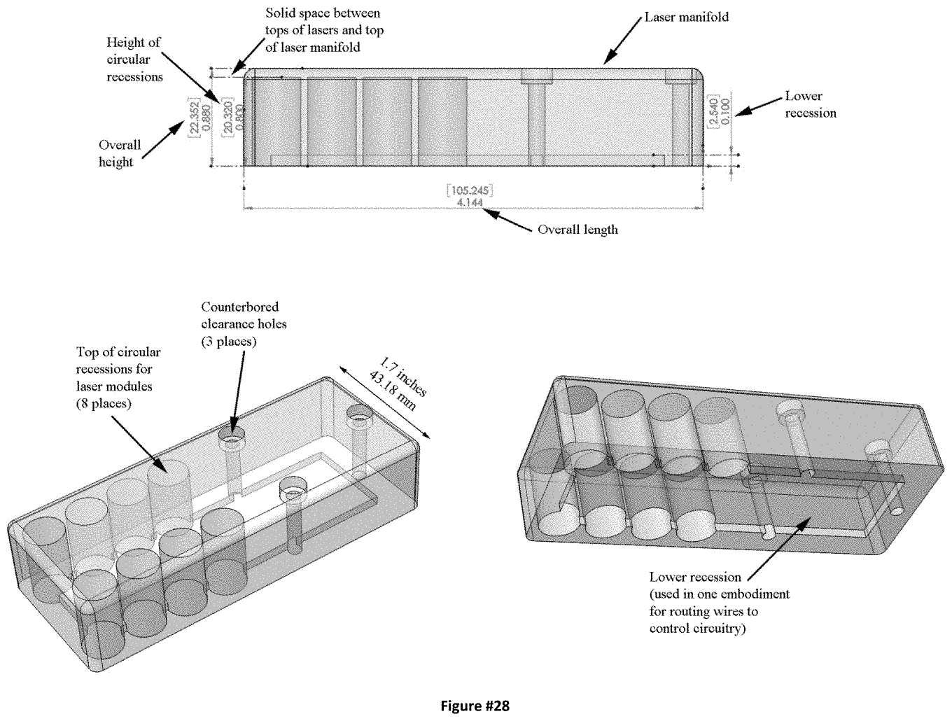

FIG. 25 demonstrates a close-up view of FIG. 24, zooming in on the distracted, or unloaded intervertebral discs of FIG. 24, the unloaded intervertebral discs shaded in black and the unloaded discs shaded in gray. The unloaded intervertebral discs extend from C2-3 to C4-5, C4-5 being the targeted disc for true decompression therapy.

Cervical Laser Traction and Cervical Laser Decompression Therapy

It is well demonstrated in the peer-reviewed, published clinical literature that wavelengths in the so-called ‘optical window’, ranging from roughly 600 nm to approximately 940 nm penetrate the skin (when applied externally to the body) and affect patient tissue and blood. The effects range from upregulating adenosine triphosphate (ATP) production, ATP being the energy currency of the body. In states of dysregulation, wound repair, fatigue and other diseases, a substantial amount of the body's normal ATP production is devoted to healing. Light therapy within these wavelength windows has been shown to be absorbed (the photonic energy) by certain chromophores, fluorophores, flavins and other structures which translates to increases in the respiratory chain which produces ATP, and thus upregulates this process and thus aids both in the healing process and the patient's perceived quality of life. Further well-studied effects of wavelengths in this region include shortened rates of known healing times, improved quality of wound healing, improved rheological properties of the blood, improved oxygen transport, upregulated immune system function, and most importantly reduction of inflammation (for example reduction of pro-inflammatory cytokines, upregulation of anti-inflammatory cytokines, and reduction of pro-inflammatory pre-cursors).

The depth of penetration of laser wavelengths tends to be shallower towards the 600 nm range than the near infrared range (900 nm). As the cervical spine is surrounded by paraspinal and other muscular tissue, connective tissue, and other tissue, an infrared wavelength may be most desirable for its depth of penetration.

The application of a laser therapy modality to the treatment of the cervical spine is a common practice. At the time of this writing of this provisional patent application, the application of cervical spinal traction or cervical spinal decompression with the simultaneous application of a laser therapy modality is uncommon and to the best of the inventors' knowledge non-existent in the medical device market/industry. In one aspect, the simultaneous application of laser therapy with either cervical traction or cervical decompression is a convenience for the healthcare provider (not having to expend billable time and effort to provide one therapy followed by another) and for the patient (not having to schedule more time to receive both therapies in series, possibly impacting their work schedule or other responsibilities). In another aspect, the simultaneous application of laser therapy and either cervical traction or cervical decompression (basic or true) offers an anatomical benefit. This anatomical benefit takes the form of improved penetration of the photonic laser energy into the intervertebral discs, once distracted. Laser penetration through bone is limited, when compared to penetration through skin/muscle/tissue/blood/etc. When the cervical spine, in its normal lordotic state, is normally loaded/not aligned or distracted lasers placed underneath the cervical spine (assuming the patient is lying supine on a treatment bed) will affect the properties discussed previously beneficial to the patient, but in a limited way due to the bony vertebra shielding in part the intervertebral discs which are to be treated by either traction, basic decompression, or true decompression. However, as with true decompression (refer to FIG. 25) the proper alignment of the skull/C0/C1/C2 (eliminating the variables associated with the unique flexion and rotation of this region), and through the rotation of the hinge plate relative to the base plate (providing alignment of the cervical vertebra from C2 through to the final vertebra to be aligned), and through a traction or true decompression (cycling of the unloading force) the extension of the vertebra and thus the intervertebral discs involved creates an improved scenario for laser penetration into the intervertebral discs, especially when laser wavelengths utilized provide deeper penetration into the affected regions. The unloaded and extended intervertebral discs also unload the nerves/nerve roots, which allows for the reduction of nociceptive signaling (the pain signal) from the site of pain which travels into the spinal cord, through to the amygdala (where pain is interpreted by the brain). The amygdala, receiving a reduced pain signal, will transmit a descending signal to the site of the “injury” (often an impinged nerve/nerve root caused by a bulging disc for example), resulting in less inflammatory pain response.

The nerves/nerve roots exit the spine on either side of center of the spine at each of the vertebra/intervertebral discs. While it may seem advantageous to place a single laser at the center of each location of treatment (within a system), it may be and is the subject of one embodiment of the present invention, to place two lasers on either side of the center of the spine. One other aspect of the laser therapy modality, applied either internally or externally which has been reported extensively in the peer-reviewed published literature is the concept of ‘translation’. Translation refers to the concept that the fluids surrounding the tissues/wounds etc. to be treated (for example blood, spinal fluid, etc.) that are exposed to an appropriate wavelength of light and an appropriate intensity of light will carry these effects to both nearby and the entire system of the patient body. Indeed, whole therapeutic modalities exist which are based on the process of translation, for example ultraviolet blood irradiation (the withdrawal of a very small amount of blood, the exposure of that blood to intense ultraviolet light [often either UVC or UVB or both], and the re-introduction of that light into the patient). Ultraviolet blood irradiation (“UBI”) has been established for well over one-hundred years, its creation resulting in a Nobel Prize for the treatment and continuously documented treatment of a variety of diseases, including sepsis and a myriad of other medical disease states.

There is a need for the integration of the laser therapy modality with cervical traction, basic cervical decompression, and most importantly and effectively true cervical decompression. The remainder of this provisional patent application will describe an apparatus

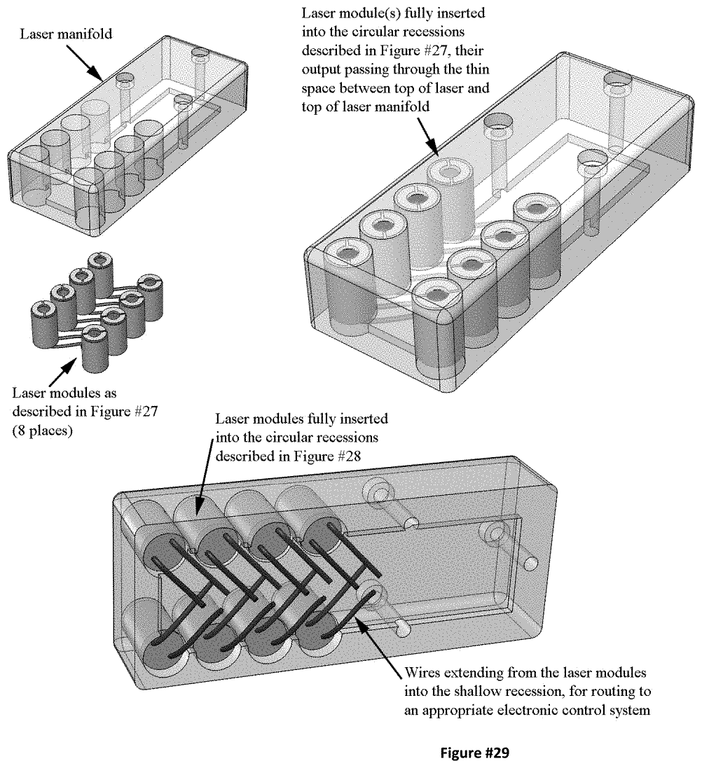

FIG. 26 demonstrates the current state of technology for solid state laser modules. Compact laser diodes of miniature size range in wavelengths from less than 635 nm to greater than 904 nm based on the state-of-the-art laser technology of today. These laser diodes tend to increase in size relative to the optical output intensity (typically given in mW). State-of-the-art laser diode technology is now placed into metallic tubes which tend to contain control circuitry and feedback mechanisms to ensure optical output is maintained. The state-of-the-art control technology allows for the simplest of power connections, namely two wire DC voltage connection, and tends to align with common logic circuit voltage of 3.3 VDC. This system of a contained laser/control circuitry/feedback circuitry in miniaturized form leads directly to one embodiment of the present invention.

In terms of physical presentation, the laser module packages range from less than 6.4 mm in diameter to greater than 10.4 mm. These modules tend to range between 12 mm to greater than 17 mm. In terms of optical output, these modules tend to put out less than 1 mW to greater than 200 mW.

There are other variations of the current state-of-the-art laser module packages, such as a small conductive spring placed at the bottom of the laser module, wherein the spring carries the positive voltage signal and the case itself carries the ground signal. However, in the embodiment of the present invention demonstrated in FIG. 26, the two wire system is utilized.

In this embodiment of the present invention, the laser module represented in FIG. 26 is an 808 nm laser wavelength. The optical power output is 50 mW. The optimal operating voltage is 3.3 volts DC (VDC). The physical package is 10.4 mm in diameter and 17 mm in length. A two wire connection is required to turn the laser on. There exists a focal lens aligned over the laser diode, and a slotted component allowing for a focusing mechanism, whereby the laser may be purely a straight beam, or slightly defocused such that instead of a single focal point some distance from the laser, a defocused region of the laser, forming a small ‘circle’ of laser light on the intended target. For example, this type of laser module is produced by US-Lasers, part number M808-50.

FIG. 27 illustrates both the focused single point laser emission as well as the left and right sides of the cone of laser light produced by slightly defocusing the laser emission.

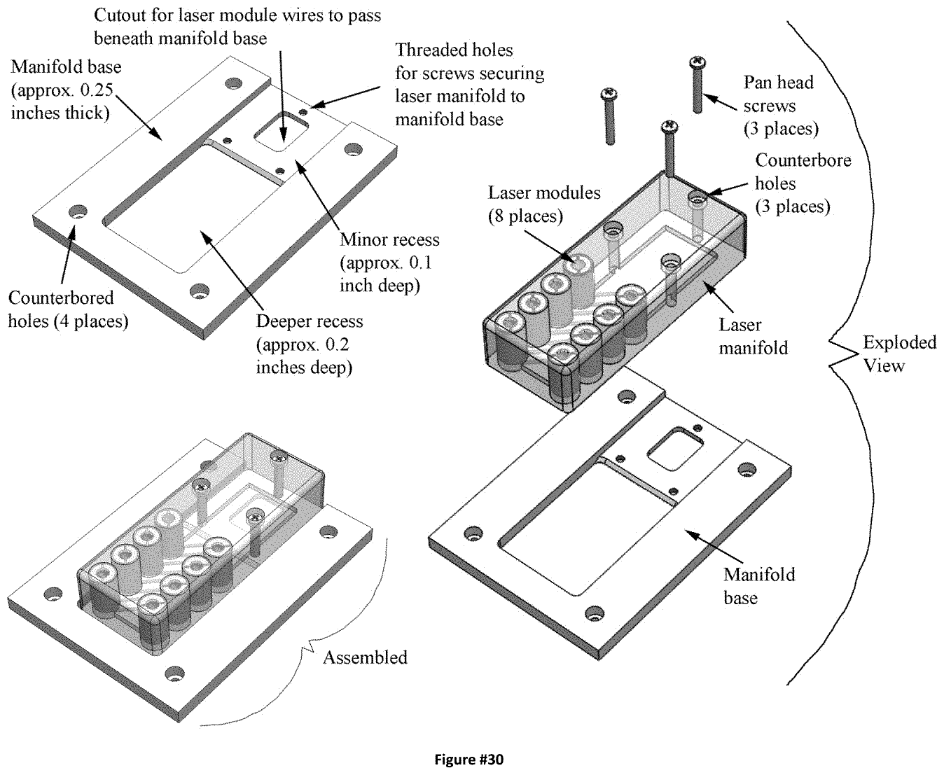

As previously described, one embodiment of the present invention involves placement of two laser modules on either side of a central axis (representing the center of the cervical spine). FIG. 28 demonstrates a “laser manifold”, a mechanical structure designed to house the laser modules and permit their optical emission to pass through to the cervical spine. Due to the state-of-the-art laser module mechanical dimensions suggested in one embodiment of the present invention, the mechanical dimensions of the laser manifold are minimal. In this embodiment, 4.14 inches in length, 0.88 inches in height, and 1.7 inches in width. Circular recessions exist which extend from the bottom of the manifold up and to 0.08 inches from the top of the laser manifold, and total eight in number. A total of 400 mW of optical energy is provided in this embodiment, derived from the eight 50 mW laser modules.

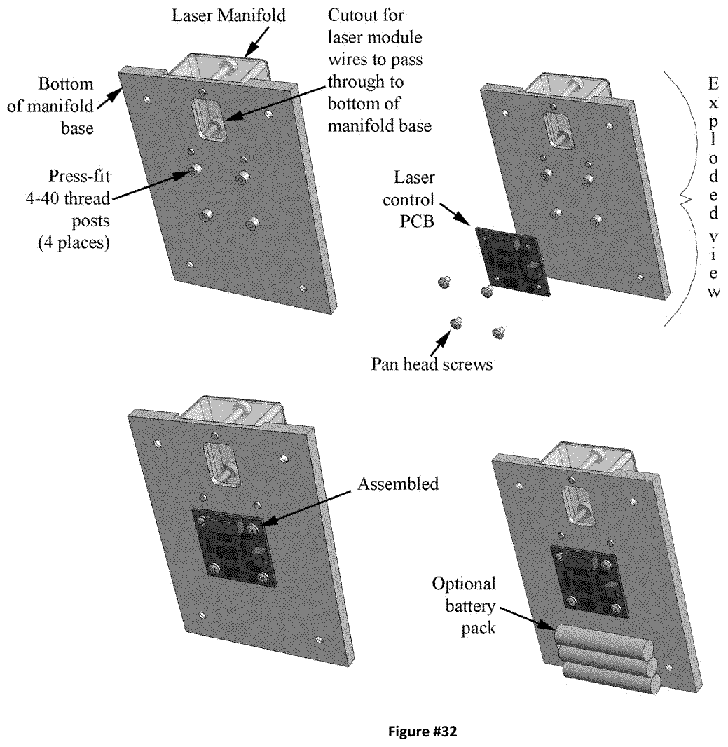

The laser manifold can be injection molded, machined, or by some other means created of a suitable material transparent to the desired wavelength(s) utilized, and in this embodiment this material would be maximally transparent to 808 nm optical energy (ideally between 80% to 100%). It is necessary to polish the space between the top of the circular laser recession and the top of the laser manifold. Three counterbored holes are shown, which can be utilized with appropriate hardware (e.g. screws) to secure the laser manifold to some type of base (shown in FIG. 30). A recession at the bottom of the manifold exists (0.10 inch in depth) which allows for routing the laser module wires to some appropriate electronic control mechanism.

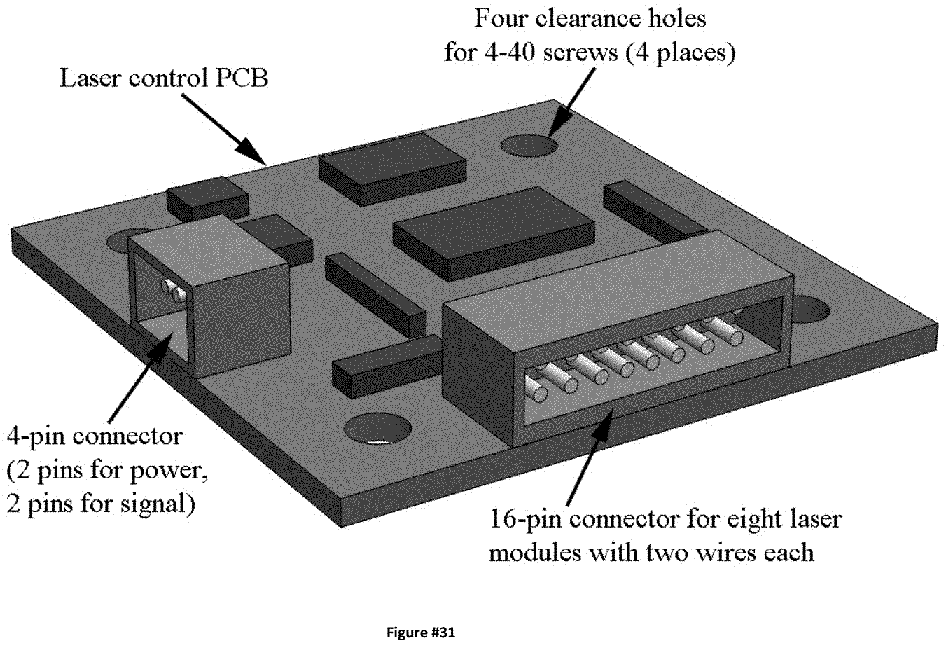

FIG. 29 illustrates an array of eight laser modules positioned below the laser manifold (top left). At top right, the laser modules are shown fully seated within their circular recessions. At the bottom of the Figure, the laser module wires are routed towards the end of the laser manifold which contains the counterbored holes and are contained within the bottom 0.1 inch recession.

FIG. 30 demonstrates an assembly which is one embodiment of the present invention. At top left, a “manifold base”, machined from a suitable, preferably lightweight material such as aluminum or delrin, is shown, which is 0.25 inches thick. A deeper recess is identified, 0.2 inches in depth, which corresponds when mated to the laser manifold the area beneath the laser modules. A minor recess is also identified which corresponds to the portion of the laser manifold which contains the three counterbored holes, and when the laser manifold is mated to the manifold base provides a foundation upon which the laser manifold is secured. Three threaded holes (in this embodiment 4-40 thread) are identified within the minor recessed region. A cutout through the entire manifold base is shown, meant to allow the laser module wires to pass through, for connection to some electronic control means. At right, an exploded view of the assembly of FIG. 30 is shown. Progressing downward from three cross-slotted pan screws, 4-40 thread and 0.875 inches long, the laser manifold is shown (which contains the laser modules) and beneath that, the manifold base. At lower right, the exploded assembly is shown collapsed and completed.

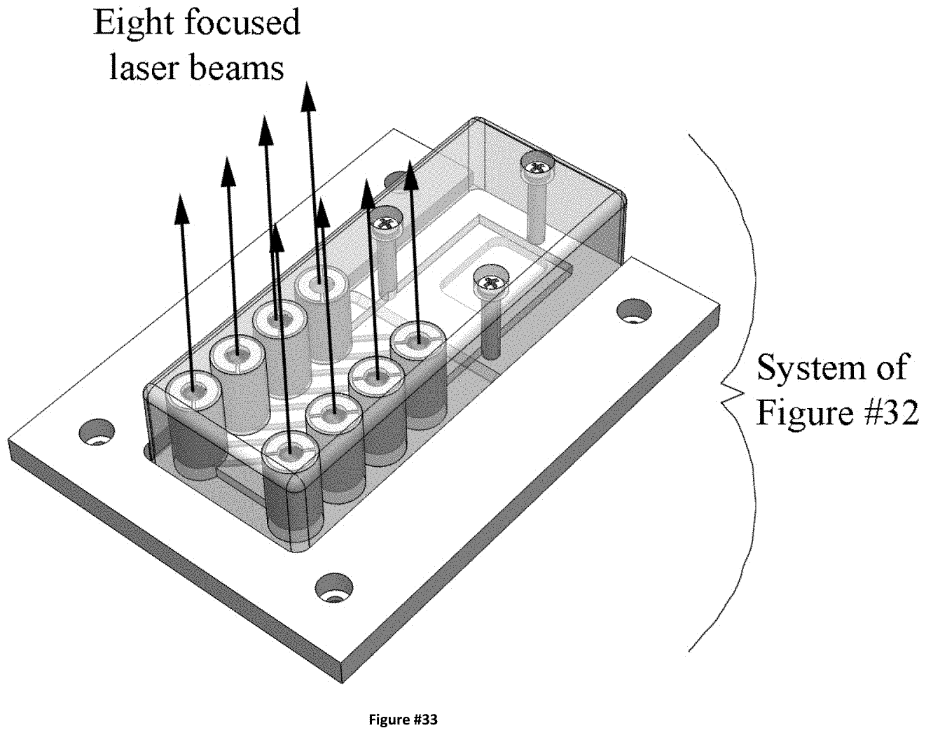

FIG. 31 illustrates an electronic means for controlling the laser modules in one embodiment of the present invention. A printed circuit board (“PCB”) is shown, the ‘laser control PCB’, which is 0.060 inches thick and measures 1.4 inches on either side. Aside from the necessary circuitry required to control the laser modules, the laser control PCB contains in this embodiment a 4-pin connector suitable to receive power (2 pins) and a control signal (2 pins). A control signal may be necessary for external communication for a variety of purposes. In one example, the control signal may be connected to a simple switch, allowing a healthcare provider to turn the laser modules on or off. In another example, the control signal may be connected to a sensor which in some way detects the appropriate conditions to turn the laser modules on or off (for example a pressure sensor). In another embodiment, the laser control PCB may be in communication with another device which, in one of the final embodiments of the present invention, controls not only when the laser modules are on or off but also controls either the traction or decompression therapy described previously in this application, as when cervical laser traction or cervical laser decompression are applied simultaneously.

The laser control PCB also contains a 16-pin connector, suitable for connection to the wires extending from the eight laser modules in the system of FIG. 30 (eight laser modules with two wires each). The laser control PCB also contains four holes suitable for clearance of #4 screws.

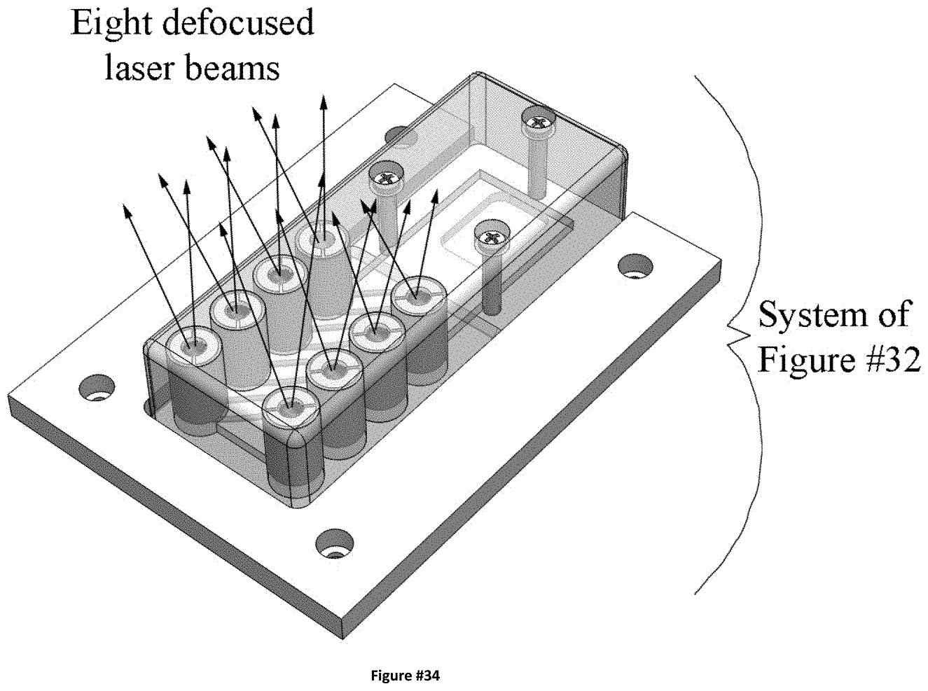

FIG. 32 describes the assembly of the system of FIG. 30, the laser control PCB, and an optional battery pack. At upper left, the bottom of the manifold base is shown, demonstrating the placement of press-fit threaded inserts which in this embodiment are 4-40 threaded and 0.1 inch tall (four places). An exploded view of the assembly is shown at top left, demonstrating four cross-slotted pan head screws (4-40 thread, 0.125 inches long), followed by the appropriate orientation of the laser control PCB, and finally the assembly shown at top left. Note the orientation of the 16-pin connector relative to the cutout in the manifold base. The wires extending from the laser modules pass through the cutout, across the bottom of the manifold base, and into the 16-pin connector.

At bottom right, the assembly is completed, the laser control PCB mated to the bottom of the manifold base via installation of the four pan head screws into the four press-fit threaded inserts. At bottom right, an optional battery pack is shown, consisting in one embodiment of the present invention three battery cells arrayed side by side (0.4 inches in diameter each and two inches long). The battery pack is centered widthwise behind the laser control PCB, 0.2 inches from the back of the manifold base. Battery packs such as described herein are commonly available, either off-the-shelf or custom made, which are commonly wrapped together, their power output routed via two wires to a common connector, and in this case potentially specifically made to attach to the power pins of the laser control PCB. In a portable application of the laser assembly of FIG. 32, batteries may provide a portable functionality. In one final embodiment of the present invention, the laser control PCB is connected via the 4-pin connector to an external device which provides both power and communication signals to the laser control PCB and which also provides cervical traction or decompression simultaneously.

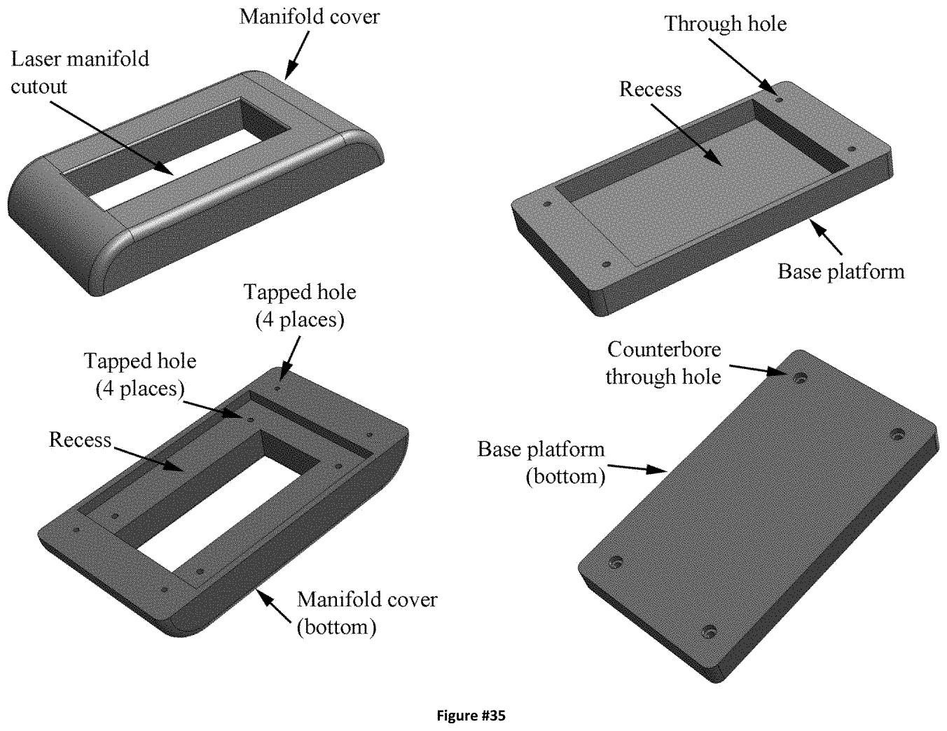

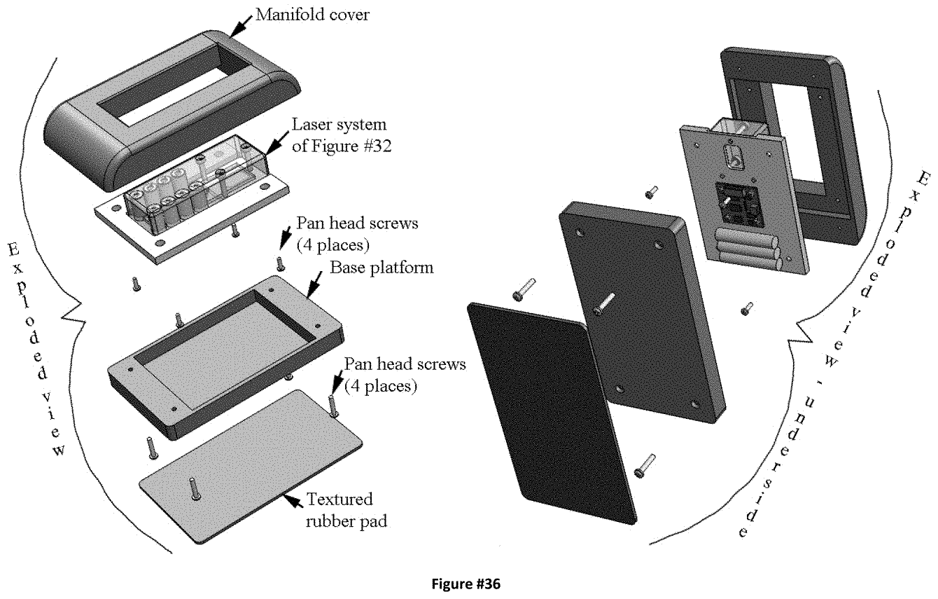

FIG. 33 describes the system of FIG. 32, with eight focused laser beams emitting from the eight laser modules installed. FIG. 34 describes the system of FIG. 32, with eight defocused laser beams emitting from the eight laser modules installed.

Although the invention has been explained in relation to its preferred embodiment(s), it is to be understood that man other possible modifications and variations can be made without departing from the spirit and scope of the invention as hereinafter claimed.

FIG. 35 describes components which can be utilized to form a portable housing for the laser system of FIG. 32. At top left, the ‘manifold cover’ is a cover upon which the laser system may be covered. In one embodiment of the present invention, the manifold cover is machined from Delrin or other non-metallic material, or may be injection molded plastic, preferably a non-metallic material. When viewed from the top, a cutout region exists allowing the top of the laser manifold to fit such that the top of the laser manifold is flush with the flat top of the manifold cover. At bottom left, the bottom of the manifold cover is shown. A secondary recession is shown, which allows the manifold base to fit flush with the bottom of the manifold cover. The bottom of the manifold cover in this embodiment of the present invention contains four tapped holes, about the outside of the bottom of the cover (in this embodiment 4-40 thread), and four tapped holes within the secondary recession (again in this embodiment 4-40 thread).

At top right, the ‘base platform’ is shown, made of similar material and manufacturing process as the manifold cover. Four through holes are shown (which are part of the four counterbore through holes shown at bottom right), as well as a recess appropriately deep to allow the laser control PCB and optional battery to reside. In this embodiment of the present invention, the manifold cover is 0.98 inches tall, 6.71 inches long and 3.8 inches wide. In this embodiment of the present invention, the base platform is 0.6 inches tall, 6.71 inches long and 3.8 inches wide, the recess being 0.5 inches deep.

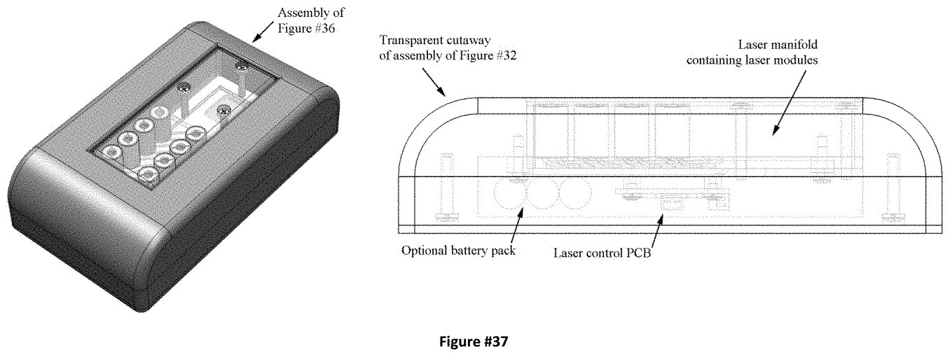

FIG. 36 illustrates exploded views of the laser system of FIG. 32 being secured into the shell formed of the manifold cover and base platform. At right, the manifold cover is shown positioned over the laser system of FIG. 32 properly oriented. Four cross-slotted pan head screws (4-40 thread, 0.375 inches long) are shown beneath the laser system of FIG. 32 which secure the laser system into the manifold cover. The manifold base then encases the manifold cover/laser system from below, secured to the manifold cover by four cross-slotted pan head screws (4-40 thread, 0.75 inches long). An optional textured rubber pad is shown, in this embodiment made of a textured silicone rubber 0.1 inches thick. This textured rubber pad may be secured via adhesive. At left, the system is shown from below.

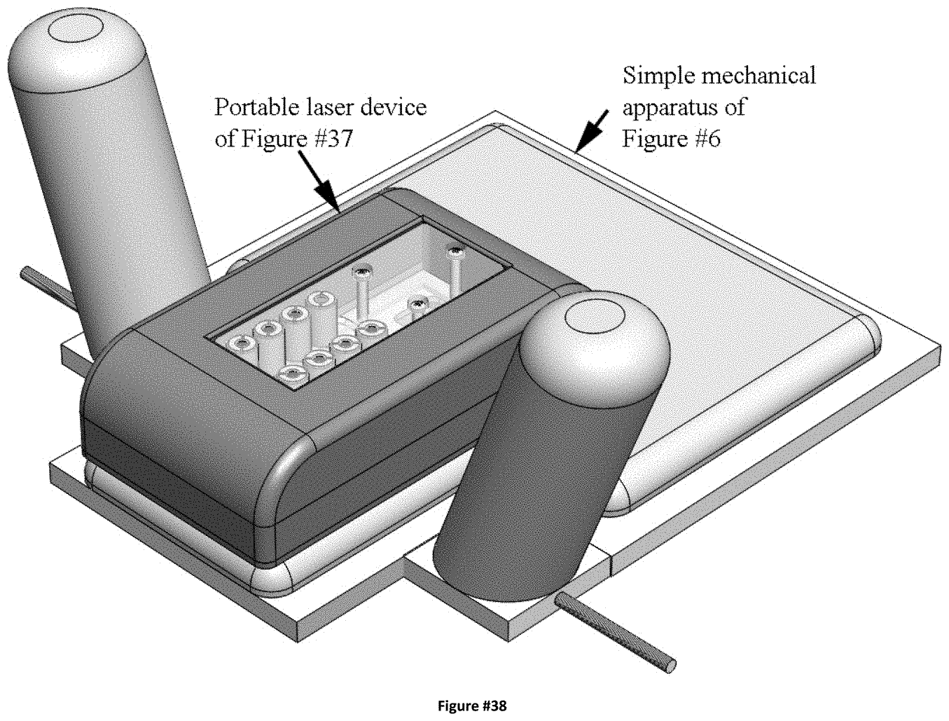

FIG. 37 shows the system of FIG. 36 fully assembled at right, the ‘portable laser device’. At left, a side view of this system is shown, in transparency such that the inner structures can be visualized within the assembly.

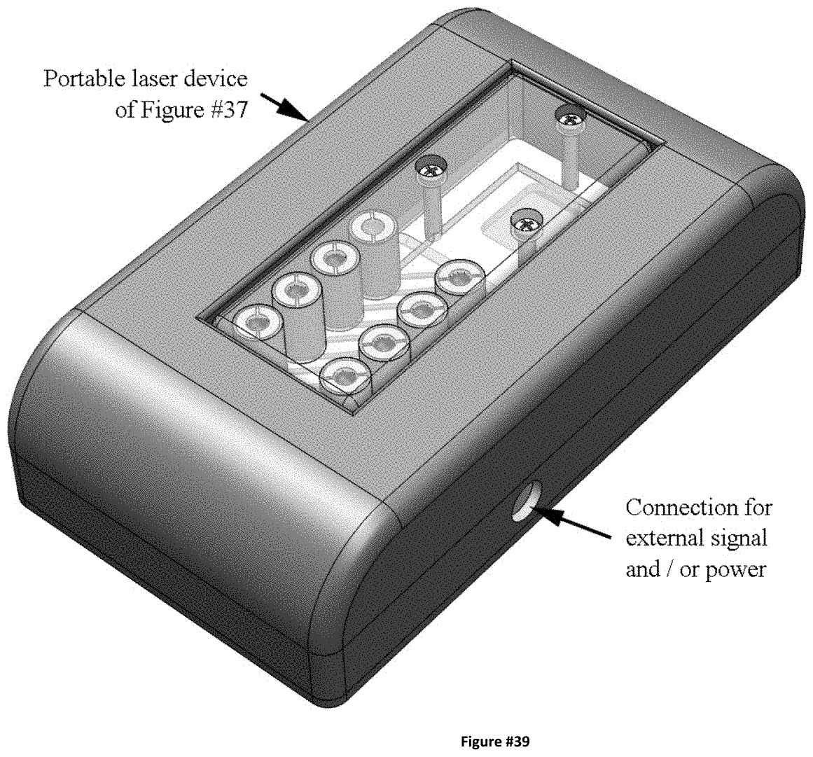

The portable laser device as described in FIG. 37 may be used in any number of environments. It may be used as a standalone laser therapy for topical wound healing, or may be placed for standalone cervical or lumbar spine treatment. FIG. 38 demonstrates the portable laser device placed appropriately on a simple mechanical apparatus (described in FIG. 6) appropriate for cervical traction (or potentially basic decompression). FIG. 38 may then describe cervical laser traction or cervical laser decompression if the two modalities are applied simultaneously.

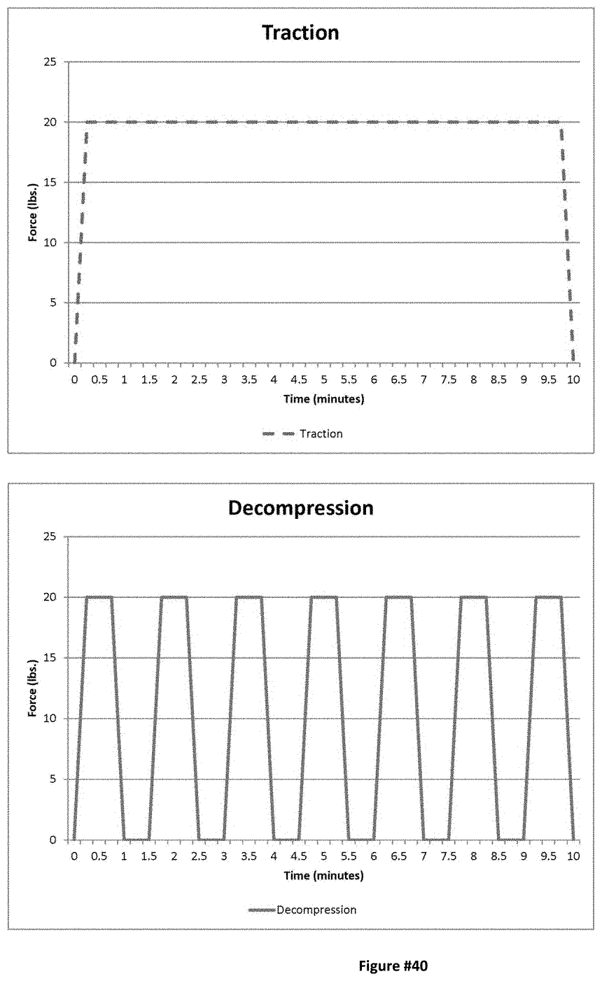

As described in FIG. 31, the laser control PCB contains a 4-pin connector designed to receive power and signal. FIG. 39 shows the portable laser device of FIG. 37, with a modification of a port or connection by which a signal may be delivered from outside of the portable laser device. In the simplest case, this may take the form of a simple on/off switch (requiring only two wire connections to two of the pins of the 4-pin connector of the laser control PCB, the other two pins of this connector being connected to the optional battery pack) such that the healthcare provider can control the laser emissions during therapy. In another form, this port may be contain a connector for both external power and communications. In the case of external power and communications, the optional battery pack is not required, reducing weight and build complexity, or is contained within the portable laser device and the external power supplied may recharge the batteries. When the portable device is connected to an external system which controls an automated traction or decompression function, the external system may then have control on when the laser emissions occur, and may coordinate these during traction or decompression to achieve either cervical laser traction or cervical laser decompression if the two modalities are applied simultaneously.

FIG. 40, referenced previously in this provisional patent application, demonstrates the profile of the forces applied during both traction and decompression therapy. At top, a graph titled ‘Traction’ shows distraction forces applied during traction (dashed line), ramping up at start of treatment and being maintained throughout the therapeutic session (y-axis is force in pounds [lbs.], x-axis is time in minutes). At bottom, a graph titled ‘Decompression’ shows distraction forces applied during either basic or true decompression. These forces ramp up at the start of treatment, are maintained for a short period of time, and then cycled down (to either a lesser distractive force or to no distractive force), the pattern repeating throughout the treatment. For either cervical laser traction or cervical laser decompression, the laser emissions may be on throughout the entire treatment, may be on only when the peak distractive forces in the case of either basic or true decompression are being applied, or in any combination thereof. Additionally, the lasers may operate in a continuous wave mode, either on or off, or the lasers may be pulsed at any appropriate frequency.

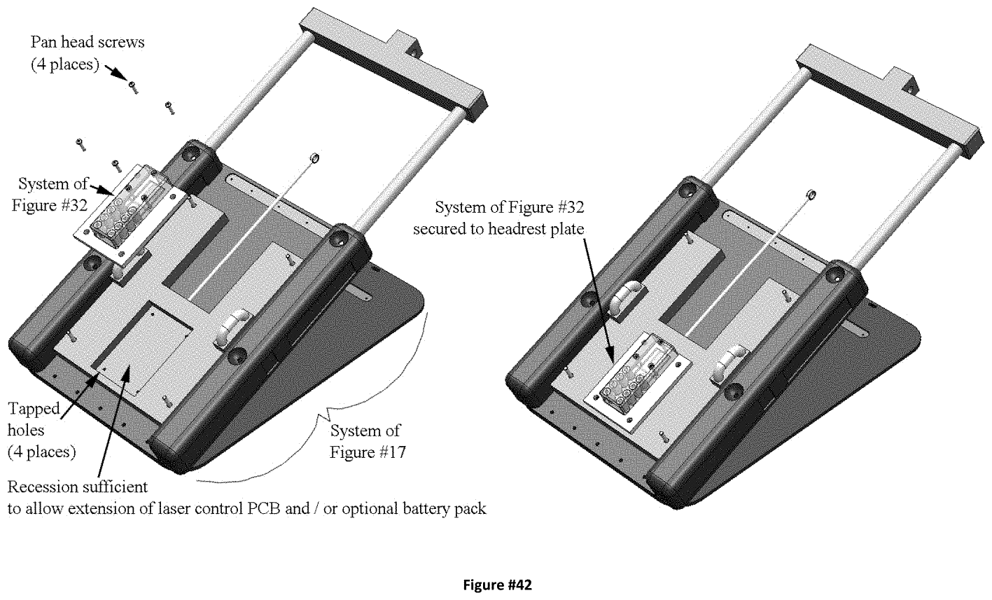

In an optimal embodiment of the present invention, the contoured headrest of FIG. 12 is shown with modification in FIG. 41. At left, two ‘laser slots’ are shown as cutting through the entire contoured headrest and positioned below C2 when the skull/C0/C1/C2 are aligned and secured properly within the contoured headrest as previously described. At right, the bottom of the modified contoured headrest is shown. Four holes are shown as were previously described for aligning and securing the modified contoured headrest to the headrest plate. The laser slots are shown going through the modified contoured headrest. A central recession is shown which conforms to the physical dimensions of the exposed portion of the laser manifold of the system described in FIG. 32. A lower recession is shown which conforms to the manifold base such that the bottom surface of the manifold base is flush with the bottom of the modified contoured headrest. In this embodiment of the present invention, the four laser modules on each side of the laser system of FIG. 32 align perfectly within the two laser slots.

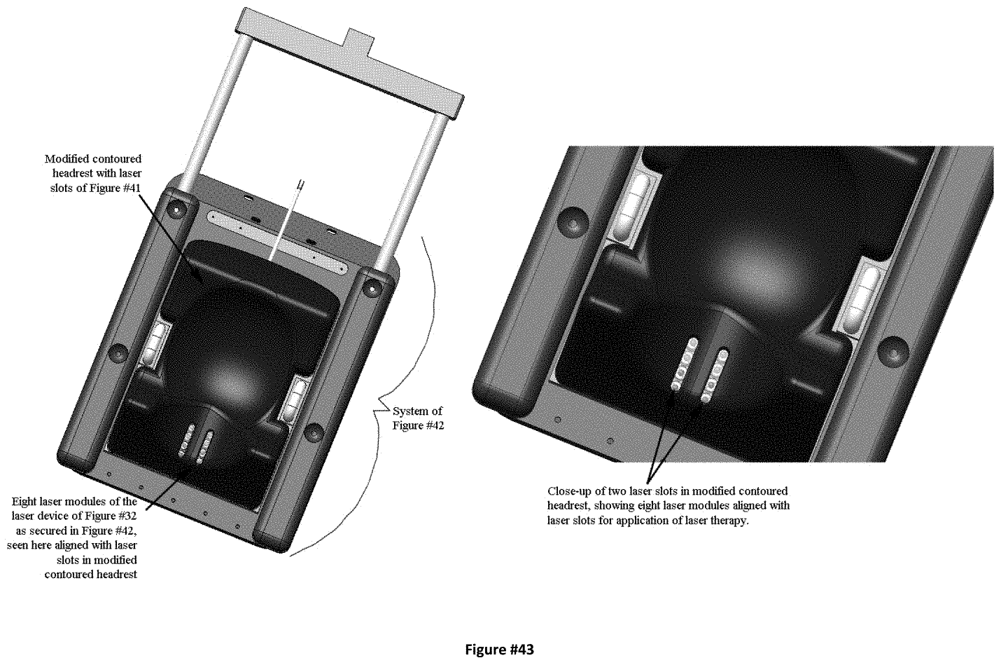

In an optimal embodiment of the present invention, FIG. 42 shows at left an exploded view of the method of securing the laser system of FIG. 32 onto the headrest plate. The system of FIG. 17 is shown at bottom, containing a recess approximately 0.5 inches deep, and containing four tapped holes (4-40 thread). The laser system of FIG. 32 is shown above the recession of the headrest plate, aligned appropriately. Four cross-slotted pan head screws, 4-40 thread and 0.625 inches in length, are shown aligned with the four holes in the manifold base.

At right, the exploded view at left is shown collapsed, the assembly completed. In this embodiment of the present invention, the laser control PCB is connected electrically through the headrest system of FIG. 17 (not shown), does not require the optional battery pack, and is supplied power and controlled by an external device which also controls the traction or decompression forces, however in this optimal embodiment of the present invention the external device likely is administering true decompression therapy. In this configuration, this optimal embodiment is then capable of providing true cervical laser decompression.

FIG. 43 shows the system of FIG. 42 with the addition of the modified contoured headrest of FIG. 41. Viewed from above, the image at left details the location of the eight laser modules centered within the two laser slots (four laser modules within each laser slot). At right, a close-up of the system is shown, to better illustrate the positioning of the laser modules relative to the two laser slots. Optimally, the most effective penetration of the laser emissions would occur when the cervical laser decompression system of FIG. 43 is used in place of the one shown in FIG. 25, when the intervertebral discs are unloaded and extended.

1. A spinal therapy device, comprising:
a contoured headrest sized and shaped to conform to a human patient's skull; and
an adjustment mechanism, having a base plate securable to an external structure and having a headrest plate secured to the contoured headrest.
